(19) United States
(12) Patent Application Publication
(10) Pub. No.: US 2003/0134789 A1
(43) Pub. Date: Jul. 17, 2003

(54) Compositions and methods for protecting tissues and cells from damage, and for repairing damaged tissues

(75) Inventor: Jeffrey G. Moore, Kennebunkport, ME (US)

Correspondence Address: HALE AND DORR, LLP, 60 STATE STREET, BOSTON, MA, 02109

(73) Assignee: Phylogix LLC, Scarborough, ME (US)

(21) Appl. No.: 10/083,936

(22) Filed: Feb. 27, 2002

Related U.S. Application Data
(60) Provisional application No. 60/271,666, filed on Feb. 27, 2001.
(60) Provisional application No. 60/302,716, filed on Jul. 3, 2001.

(51) Int. Cl. A61K 38/17

(52) U.S. Cl. 514/12

(57) ABSTRACT

Disclosed are methods and compositions for protecting cells and tissue from damage, particularly damage induced by a cytotoxic agent or a therapeutic treatment. The methods include contacting a progenitor cell with a member of the FRIL family of progenitor cell preservation factors.

Also disclosed are methods for protecting normal cells and tissues in an animal from cytotoxicity induced by a therapeutic treatment, such as chemotherapy or radiotherapy. These methods include administering a FRIL family member molecule to the animal receiving the therapeutic treatment, wherein the normal cells and tissues of the animal administered the FRIL family member are protected from the therapeutic treatment's cytotoxicity.

Also disclosed are methods for isolating a cell for repairing a tissue. The methods include contacting a population of cells with a FRIL family member molecule and isolating a cell specifically bound by the FRIL family member molecule, wherein the cell bound to the FRIL family member molecule is useful for repairing a tissue.

### CROSS-REFERENCE TO RELATED APPLICATIONS

This application claims benefit from U.S. Provisional Application Serial No. 60/271,666 filed Feb. 27, 2001, and from U.S. Provisional Application Serial No. 60/302,716 filed Jul. 3, 2001, the entire contents of each of which are hereby incorporated by reference.

### BACKGROUND OF THE INVENTION

The invention relates to protecting cells and tissue from damage, and to repairing damaged tissue.

Tissues often do not recover from damage by stroke, heart attacks, spinal cord injury, acute liver failure, burns, and other acute insults. Destruction of tissue also results from chronic degenerative disease. The field of tissue engineering has focused on costly methods for repairing tissues by either developing in vitro systems designed to grow specific organs and tissues or by transplanting cells capable of repairing tissue in vivo.

Commonly used cancer therapeutics include chemotherapeutic and radiotherapeutic drugs. Radiotherapy and chemotherapy drugs work by interrupting the process of forming new cells. While these drugs selectively target rapidly growing cancer cells, they also adversely affect active normal tissues such as the bone marrow, gastrointestinal tract, and hair follicles.

Indeed, suppression of bone marrow function by chemotherapy is the most serious side-effect because it damages the progenitors needed to produce the blood cells that carry oxygen to tissues (red cells), fight infection (white cells), and clot blood (platelets). Chabner and Myers teach that reduced blood counts increase patients' susceptibility to life-threatening infections and bleeding (Chabner, B. A. and Myers, C. E., “Antitumor antibiotics,” In Cancer, Principles and Practice in Oncology, eds. DeVita, Jr. et al., Lippincott, Pa. pp. 374-384, 1993). Accordingly, delayed or incomplete restoration of blood cell production following chemotherapy not only compromises patients' abilities to fight infection but also delays subsequent administration of chemotherapy to treat growth of residual tumor cells.

Moreover, chemotherapy's effectiveness in eradicating cancer depends in part on the ability to manage toxicity to the bone marrow and gastrointestinal tract. Toxicity to the gastrointestinal tract commonly results in appetite loss and weight loss, nausea and vomiting, inflammation and sores in the mouth and throat, and bouts of diarrhea and constipation during the course of chemotherapy.

Strategies to develop supportive care products that specifically protect normal tissues from the toxicity of chemotherapy have focused on agents that either counteract the mechanism of the chemotherapeutic or reduce the proliferation rate of normal, rapidly dividing tissues. Two of these supportive care products, called chemoprotective agents, are currently either in clinical trials or in the market. Ethyol™ (amifostine), is a low molecular weight compound first identified by its ability to protect laboratory animals from lethal radiation. Mirostatin™ (Myeloid Progenitor Proliferation Factor-1 or MPIF-1), is a beta chemokine that protects myeloid (white blood cell) progenitors preventing these cells from entering an actively dividing state during chemotherapy. However, the current supportive care drugs do not adequately reduce the toxicity of chemotherapy. Ethyol™ itself induces serious side-effects of nausea and vomiting and consequently is not widely used. In addition, like other chemokines, Mirostatin™, is a short-lived regulator, is likely pleiotropic (i.e., acts on many different tissues), and likely toxic at high levels. The value of Mirostatin™ is further diminished by its restriction only to mature myeloid (i.e., white blood cell) progenitors. To date, no chemoprotective agent or radiotherapeutic agent protects the patient being administered the agent from cachexia.

Thus, there remains a need for improved reagents that are non-toxic and inexpensive to produce for use in protecting normal cells and tissues against tissue damage, particularly tissue susceptible to damage and/or damaged due to the adverse effects of chemotherapeutic and/or radiotherapeutic drugs, including cachexia.

### SUMMARY OF THE INVENTION

The invention provides compositions and methods for protecting healthy cells and tissue from damage, and for repairing tissues that have been damaged. The compositions of the invention comprise at least one member of the FRIL family of progenitor cell preservation factors. Members of the FRIL family are non-toxic and inexpensively produced.

Accordingly, in a first aspect, the invention provides a method for protecting a progenitor cell against a cytotoxic agent comprising contacting the progenitor cell with a FRIL family member molecule and the cytotoxic agent, wherein the contacted cell is protected against cytotoxicity by the cytotoxic agent. Preferably, the FRIL family member molecule is purified. In certain embodiments, the progenitor cell is in a tissue. In some embodiments, the progenitor cell is a hematopoietic progenitor cell. In certain embodiments, the progenitor cell is a mesenchymal progenitor cell, a hematopoietic stem cell, a hair follicle progenitor cell, a skin progenitor cell, a liver progenitor cell, or a gastrointestinal progenitor cell.

In certain embodiments of the first aspect of the invention, the progenitor cell is in an animal, including, without limitation, a domesticated animal or a human. In certain embodiments where the progenitor cell is in an animal, the progenitor cell is contacted by administering the FRIL family member molecule to the animal. In some embodiments, the FRIL family member molecule is administered to the animal with a pharmaceutically acceptable carrier.

In various embodiments of the first aspect of the invention, the cytotoxic agent is selected from the group consisting of a chemotherapeutic and a radiotherapeutic. In some embodiments, the progenitor cell is contacted with the FRIL family member molecule before the cell is contacted with the cytotoxic agent. In some embodiments, the progenitor cell is contacted with the FRIL family member molecule after the cell is contacted with the cytotoxic agent.

In a second aspect, the invention provides a method for protecting a progenitor cell in a patient against a progenitor cell-depleting activity of a therapeutic treatment in a patient, comprising administering a therapeutically effective amount of a FRIL family member molecule to the patient with the therapeutic treatment, wherein the progenitor cell in the patient is protected against the progenitor cell-depleting activity of the therapeutic treatment. In this method, normal progenitor cells are protected against the progenitor cell-depleting activity of therapeutic treatments. In some embodiments, the patient is a domesticated animal. Preferably, the patient is a human. In some embodiments, the patient has cancer.

In certain embodiments of the second aspect, the therapeutic treatment is a radiotherapeutic, a chemotherapeutic, or a combination of a radiotherapeutic and a chemotherapeutic. In certain embodiments, the chemotherapeutic is cytarabine, doxorubicin, cisplatin, daunorubicin, paclitaxel, cyclophosphamide, or 5-fluorouracil. In certain embodiments, the FRIL family member molecule is purified. In certain embodiments, the FRIL family member molecule is administered to the patient with a pharmaceutically acceptable carrier. In some embodiments, the patient is administered the FRIL family member molecule before administration of the therapeutic treatment.

In a third aspect, the invention features a method for isolating a cell for repairing a tissue comprising contacting a population of cells with a FRIL family member molecule and isolating a cell specifically bound by the FRIL family member molecule, wherein the cell bound to the FRIL family member molecule is useful for repairing a tissue. In certain embodiments, the population of cells is from a human, the population of cells includes a progenitor cell, and the population of cells is whole blood, umbilical cord blood, fetal liver cells, or bone marrow cells.

In certain embodiments of this aspect of the invention, the cell bound by the FRIL family member molecule is a progenitor cell. In certain embodiments, the progenitor cell is a messenchymal progenitor cell, a hematopoietic stem cell, a hair follicle progenitor cell, a skin progenitor cell, a liver progenitor cell, or a gastrointestinal progenitor cell.

According to the invention, compositions of a FRIL family member may be used as therapeutic agents to prevent the damage of healthy tissue in patients, such as cancer patients receiving chemotherapy. For example, a FRIL family member may be administered with a pharmaceutically-acceptable carrier (e.g., physiological sterile saline solution) via any route of administration to a cancer patient receiving chemotherapy in an attempt to reduce the healthy tissue damaging effects of the chemotherapeutic so that the patient can receive a higher dose of the chemotherapeutic and, preferably, recover from cancer. Pharmaceutically-acceptable carriers and their formulations are well-known and generally described in, for example, Remington's Pharmaceutical Sciences (18th Edition, ed. A. Gennaro, Mack Publishing Co., Easton, Pa., 1990).

### BRIEF DESCRIPTION OF THE DRAWINGS

FIGS. 1A-1D are representations of flow cytometry histograms of FRIL-binding umbilical cord blood cells isolated according to the invention. FIG. 1 shows the relative size of the cells, wherein the encircled area (“gated”) represent the live cells. FIG. 1B shows that only 3% of the gated cells positively stained with non-specific IgG1 antibody labeled with phycoerythrin (PE). FIG. 1C shows that 50% of the gated cells positively stained with PE-labeled anti-FLT3 antibody. FIG. 1D shows that 60% of the gated cells positively stained with PE-labeled anti-KIT antibody.

FIGS. 2A and 2B are representations of flow cytometry histograms of FRIL-binding umbilical cord blood cells isolated according to the invention. FIG. 2A shows that of the cells that do not stain positive with PE-labeled non-specific antibody, only 0.06% positively stained for FITC-labeled anti-CD38 antibody. FIG. 2B shows that of the cells that stained positive with PE-labeled anti-FLT3 antibody, 64% of the cells did not express CD38, while 36% of the cells did express CD38 (i.e., 36% positively stained for FITC-labeled anti-CD38 antibody).

FIGS. 3A and 3B are representations of line graphs showing the response of FRIL-binding umbilical cord blood cells, isolated according to the invention, to endothelial or hematopoietic stimuli. FRIL-binding cells (“FRIL+”) formed a large number of colonies in the presence of either endothelial (FIG. 3A) or hematopoietic (FIG. 3B) stimuli. In contrast, unsorted umbilical cord blood cells (“CB”) and mononuclear cord blood cells that did not specifically bind to FRIL (“FRIL−”) formed fewer numbers of colonies in the presence of either endothelial (FIG. 3A) or hematopoietic (FIG. 3B) stimuli.

FIG. 4 is a representation of a polymerase chain reaction analysis of mRNA extracted from umbilical cord blood mononuclear cells (wells labeled “1”), FRIL binding umbilical cord cells isolated according to the invention (wells labeled “2”) or CD34 binding umbilical cord blood cells isolated according to the invention (wells labeled “3”) amplified with primers that specifically bind to the GAPDH MRNA (left three lanes) FLT3 receptor MRNA (middle three lanes) or FLT1 receptor mRNA (right three lanes).

FIGS. 5A-5D are representations of line graphs showing the dose response of CB mnc chemotherapeutic agents in the presence and absence of Dl-FRIL, a representative, non-limiting FRIL family member of the invention. Chemotherapy agents were assayed over a 5-log dose range on CB MNC (2×10⁵ cells /0.1 mL) in AIMV (Life Technologies) containing 10% Agar-SCM (StemCell Technologies). Solid circles indicate chemotherapy drug with no Dl-FRIL; solid triangles indicate cultures containing Dl-FRIL at 10 ng/ml in all wells; and open circles indicate Dl-FRIL in all wells at 100 ng/ml. FIG. 5A shows the dose response to Ara-C; FIG. 5B shows the dose response to cisplatin; FIG. 5C shows the dose response to doxorubicin, and FIG. 5D shows the dose response to 5-FU.

FIGS. 6A-6C are schematic representations of various non-limiting protocols for timing the administration of a Dl-FRIL, a representative, non-limiting FRIL family member of the invention, compared to the administration of 5-FU. In FIG. 6A (“pre-treatment with FRIL”), administration of four doses of Dl-FRIL occurs daily starting three days prior to the first day that 5-FU is administered. In FIG. 6B (“FRIL during 5-FU treatment”), administration of five doses of Dl-FRIL starts the day before the first administration of 5-FU and continues through the second administration of 5-FU. In FIG. 6C (“optimized FRIL administration”), four doses of Dl-FIL are administered, where each Dl-FRIL administration occurs the day before and the day of administration of 5-FU.

FIG. 7 is a survival graph showing the increased survival of 5-FU treated animals administered 100 μg DI-FRIL, a representative, non-limiting FRIL family member of the invention (open circles) or Hank's Balanced Salt Solution (HBSS; closed circles). In the lower left of FIG. 7 are the dosage schedules of Dl-FRIL and 5-FU.

FIG. 8 is a survival graph showing the increased survival of 5-FU treated animals administered the indicated amounts of Dl-FRIL, a representative, non-limiting FRIL family member of the invention as compared to those animals administered HBSS (closed circles). In the lower left of FIG. 8 are the dosage schedules of Dl-FRIL and 5-FU.

FIGS. 9A and 9B are line graphs showing the body weights of 5-FU treated mice administered Dl-FRIL, a representative, non-limiting FRIL family member of the invention (FIG. 9B) or Hank's Balanced Salt Solution (HBSS) (FIG. 9A). In the lower left of FIGS. 9A and 9B are the dosage schedules of HBSS and 5-FU (FIG. 9A) and Dl-FRIL and 5-FU (FIG. 9B).

FIG. 10 is a representation of a flow cytometry histogram of CD34+ cells stained with propidium iodide the day they were sorted. As can be seen, 96.6% of the cells are in the non-cycling G₀/G₁ stage of the cell cycle.

FIGS. 11A and 11B are bar graphs showing the numbers of viable cells (FIG. 11A) and progenitors (FIG. 11B) after incubation of CB CD 34+ cells in cells incubated with FRIL, washed, and subsequently incubated with either FRIL (gray bars) or cytokine cocktail (black bars) for the number of days indicated.

### DETAILED DESCRIPTION OF THE PREFERRED EMBODIMENTS

The invention provides compositions and methods for preventing cell and tissue damage, particularly cell and tissue damage resulting during treatment with a chemotherapeutic or radiotherapeutic. All of the patents and publications cited herein reflect the knowledge in the art and are hereby incorporated by reference in entirety to the same extent as if each were specifically stated to be incorporated by reference. Any inconsistency between these patents and publications and the present disclosure shall be resolved in favor of the present disclosure.

In a first aspect, the invention provides a method for protecting a progenitor cell against a cytotoxic agent comprising contacting the progenitor cell with a FRIL family member molecule and the cytotoxic agent, wherein the contacted cell is protected against cytotoxicity by the cytotoxic agent.

The term, “FRIL family of progenitor cell preservation factors” is used to mean a family of lectins, wherein each FRIL family member molecule preserves progenitor cells, and wherein each FRIL family member molecule binds to a normally glycosylated FLT3 receptor (see Moore et al., Biochim. Biophys. Acta 25027: 1-9, 2000). By “lectin” is meant a protein that binds sugar residues with high affinity. In accordance with the first aspect of the invention, the terms “bind,” “binds,” or “bound” are used interchangeably to mean that a FRIL family member molecule of the invention binds to a normally glycosylated FLT3 receptor with an affinity at least as high as, and preferably higher than the affinity with which the FLT3-Ligand binds the normally glycosylated FLT3 receptor. Preferably, a FRIL family member molecule binds to a normally glycosylated FLT3 receptor with an affinity that is at least as high as the affinity with which an antibody binds its specific ligand. Even more preferably, a FRIL family member molecule of the invention binds to a normally glycosylated FLT3 receptor with an affinity that is higher than the affinity with which an antibody binds its specific ligand. Still more preferably, a FRIL family member molecule of the invention binds to a normally glycosylated FLT3 receptor with a dissociation constant (K_{D}) of at least 10⁻⁷ M, more preferably 10⁻⁸ M, even more preferably 10⁻⁹ M, still more preferably, at least 10⁻¹⁰ M, and most preferably, a FRIL family member molecule of the invention binds to a normally glycosylated FLT3 receptor with a dissociation constant (K_{D}) of at least 10⁻¹¹ M. Standard methods for determining binding and binding affinity are known.

In accordance with the invention, by “normally glycosylated FLT3 receptor” is meant an FLT3 receptor that has a glycosylation pattern of an FLT3 receptor glycosylated by a normal cell. By “normal cell,” as used herein in accordance with all aspects of the present invention, is meant a living cell that is not a neoplastic cell. As used herein, by “neoplastic cell” is meant a cell that shows aberrant proliferation, particularly increased proliferation, that is not regulated by such factors as cell-cell contact inhibition and soluble regulators (e.g., cytokines or hormones), and that abnormally glycosylates the FLT3 receptor such that the glycosylation pattern on the FLT3 receptor on the neoplastic cells is abnormal and different from the glycosylation pattern of the FLT3 receptor on normal cells. Thus, the FLT3 receptor on a neoplastic cell is not bound by a FRIL family member molecule of the invention.

By “FRIL family member” or “FRIL family member molecule” is meant one or more molecules of the FRIL family of progenitor cell preservation factors. Preferably, a FRIL family member is from a legume, such as the garden pea or the common bean. Legumes are plants (“leguminous plants”) from a family (Leguminosae) of dicotyledonous herbs, shrubs, and trees bearing (nitrogen-fixing bacteria) nodules on their roots. Preferably, a FRIL family member is from members of the tribe Phaseoleae including, without limitation, Phaseolus vulgaris, Dolichos lab lab, Sphenostylis stenocarpa, Vigna sinensis, or Voandzeia subterranea. Preferably, a FRIL family member molecule is a mannose/glucose-specific legume lectin. (See Moore et al., Biochim. Biophys Acta 25027: 1-9, 2000; Colucci et al., Proc. Natl. Acad. Sci. USA 96: 646-650, 1999; Mo et al., Glycobiology 9: 173-179, 1999; and Hamelryck et al., J. Molec. Biol. 299: 875-883, 2000). Preferably, the FRIL family member molecule that is isolated from a hyacinth bean (i.e., Dolichos lab lab) has an amino acid sequence which comprises the following eight amino acid sequence: TNNVLQXT. A FRIL family member of the invention is preferably encoded by a nucleic acid molecule comprising or consisting of the sequence of SEQ ID NO:1, SEQ ID NO:3, SEQ ID NO:5 or SEQ ID NO:7. Preferably, the FRIL family member molecule of the invention has an amino acid sequence comprising or consisting of the sequence of SEQ ID NO:2, SEQ ID NO:4, SEQ ID NO:6, SEQ ID NO:8, SEQ ID NO:9, or SEQ ID NO:10.

Other molecules falling into the definition of a FRIL family member molecule (e.g., mutants or fusion proteins), as well as recombinant FRIL family member molecules, methods for making and purifying such FRIL family member molecules, and methods for purifying nucleic acid molecules encoding such FRIL family member molecules are described in U.S. Pat. No. 6,084,060; Colucci et al., PCT Application No. PCT/US99/31307 (PCT Publication No. WO01/49851), and Colucci et al., PCT Application No. PCT/US98/13046 (PCT Publication No. WO98/59038), the entire disclosures of all of which are hereby incorporated by reference.

Preferably, each FRIL family member molecule binds to a normally glycosylated FLT3 receptor and has at least about 45% amino acid sequence identity with the amino acid sequence of another member of the FRIL family, preferably at least about 55% identity, still more preferably at least about 65% identity, still more preferably at least about 75% identity, yet more preferably at least about 85% identity, and more preferably at least about 95% identity with the amino acid sequence of another member of the FRIL family (e.g., SEQ ID NO:2, SEQ ID NO:4, SEQ ID NO:6, SEQ ID NO:8, SEQ ID NO:9, or SEQ ID NO: 10). Amino acid sequence identity and nucleic acid sequence identity between two proteins or two nucleic acid molecules can be measured according to standard methods (see, e.g., Pearson and Lipman, Proc. Natl. Acad. Sci. USA 85:2444-2448, 1988; George, D. G. et al., in Macromolecular Sequencing and Synthesis, Selected Methods and Applications, pps. 127-149, Alan R. Liss, Inc. 1988; Feng and Doolittle, Journal of Molecular Evolution 25: 351-360, 1987; and Higgins and Sharp, CABIOS 5: 151-153, 1989; and the various BLAST programs of the National Center for Biotechnology, National Library of Medicine, Bethesda, Md).

Preferably, the normally glycosylated FLT3 receptor to which a FRIL family member binds is expressed on a progenitor cell. As used herein, “progenitor cell” refers to any normal somatic cell that has the capacity to generate fully differentiated, functional progeny by differentiation and proliferation. Preferably, the progenitor cell is in a tissue. Progenitor cells include progenitors from any tissue or organ system, including, but not limited to, blood, mesenchymal, hair, embryonic, nerve, muscle, skin, gut (i.e., gastrointestinal), bone, kidney, liver, pancreas, thymus, and brain. Thus, in some embodiments of the invention, the progenitor cell is a hematopoietic progenitor cell. In certain embodiments, the progenitor cell is a messenchymal progenitor cell, a hematopoietic stem cell, a hair follicle progenitor cell, a skin progenitor cell, a muscle progenitor cell, a nerve progenitor cell, a brain progenitor cell, a bone progenitor cell, a kidney progenitor cell, a liver progenitor cell, or a gastrointestinal progenitor cell.

In certain preferred embodiments of the invention, the progenitor cell expressing a normally glycosylated FLT3 receptor (to which a FRIL family member binds) also expresses the KIT receptor. The FLT3 receptor is expressed on various stem and progenitor cells in the bone marrow and dispersed throughout the tissues including, without limitation, embryonal cells, hematopoietic cells, messenchymal cells, endothelial cells, and brain cells. In certain preferred embodiments, the progenitor cell expressing a normally glycosylated FLT3 receptor (to which a FRIL family member binds) does not express the CD34 cell surface receptor.

Thus, in accordance with the invention, a FRIL family member attached to a solid support (e.g., a magnetic bead) is useful for capturing a FLT3+, Kit+, CD34− progenitor cell (see Example below). Where the solid support is a magnetic bead, the unbound cells are separated by applying a magnet to the population of cells contacted with the FRIL family member molecules immobilized on the magnetic bead and rinsing off the unbound cells. Methods for isolating progenitor cells that bind FRIL family member molecule-coated magnetic beads are described in the examples below. Magnetic beads are commercially available (e.g., from Dynabeads Tosylactivated, Lake Success, N.Y.; or from Miltenyi Biotec, Auburn, Calif.). Since the FRIL family member is protein, it can be conjugated to a magnetic bead via amino- or sulfhydryl-groups of the protein.

Preferred magnetic beads to which FRIL family member molecules are immobilized are the MACS super-paramagnetic MicroBeads (commercially available from Miltenyi Biotec, Inc., Auburn, Calif.) or M-280 Dynabeads Tosylactivated (commercially available form Dynal Biotech, Inc., Lake Success, N.Y.). Preferably, the magnetic beads are biodegradable, so that bead-bound progenitor cells retain their physiological function.

In accordance with the invention, a FRIL family member molecules may be directly or indirectly attached to the bottom of a tissue culture plate. Following a standard “panning” protocol (see, e.g., Stengelin et al., EMBO J. 7(4): 1053-1059, 1988; Aruffo and Seed, Proc. Natl. Acad. Sci. USA 84(23): 8573-8577, 1987), a population of cells suspected of containing a FRIL family member-binding progenitor cell is incubated on the plate. The plate is then gently rinsed with a physiologically acceptable solution, thereby removing the unbound cells while leaving the FRIL family member-binding population of progenitor cells attached to the FRIL family member-coated plate.

Progenitor cells are distinguished from differentiated cells, the latter being defined as those cells that may or may not have the capacity to proliferate, i.e., self-replicate, but that are unable to undergo further differentiation to a different cell type under normal physiological conditions. Progenitor cells include all the cells in a lineage of differentiation and proliferation prior to the most differentiated or the fully mature cell. Thus, for example, progenitors include the skin progenitor in the mature individual. The skin progenitor is capable of differentiation to only one type of cell, but is itself not fully mature or fully differentiated, and thus is included in the definition of a progenitor cell. Moreover, progenitor cells are further distinguished from abnormal cells such as neoplastic cells, as defined herein. Standard methods for detecting progenitor cells may be employed for determining the ability of a FRIL family member to preserve progenitor cells (e.g., the methylcellulose or other semi-solid medium based progenitor cell assay described in, e.g., Paige et al., Eur. J. Immunol. 14(11):979-987, 1984; Iscove et al., J. Immunol. 142(7):2332-2337, 1989; U.S. Pat. No. 6,084,060).

As used herein, by “preserves progenitor cells” is meant an ability of a FRIL family member (or mutant thereof or fusion protein comprising a FRIL family member or mutant thereof) to retain (i.e., preserve) progenitor cells in an undifferentiated state, which can be determined using known assays (see, e.g., Kollet et al., Exp. Hematol. 28: 726-726, 2000; U.S. Pat. No. 6,084,060). Preferably, a FRIL family member (in the absence of other cytokines) preserves progenitor cells in a dormant state for up to a month in suspension culture in serum-defined medium (see, e.g., Colucci et al., Proc. Natl. Acad. Sci. USA 96: 646-650, 1999). A non-limiting mechanism by which a FRIL family member preserves progenitor cells is by acting on FLT3-expressing progenitor cells (progenitors of, e.g., blood vessels, blood, mucosal membranes, liver, kidneys) to prevent their proliferation and secretion of regulators.

Thus, in accordance with the present invention, a FRIL family member molecule is useful for preserving progenitor cells; however, by maintaining the progenitor cells in a quiescent state, a FRIL family member is able to preserve the progenitor cells for eventual progression in the absence of the FRIL family member. For example, if a progenitor cell is placed in culture with a FRIL family member and the culture is left undisturbed, eventually, all of the FRIL family member in the culture will be used by the cells, and the cells will come out of their quiescent state. This quiescent state held by a progenitor cell when cultured in the presence of a FRIL family member insures slow cell turnover by the quiescent progenitor cells, and slows differentiation by those quiescent progenitor cells. Non-limiting mechanisms by which a FRIL family member acts includes, for example, maintaining a progenitor cell's viability, preventing (or slowing) a progenitor cell's progression toward differentiation, and/or maintaining the a progenitor cell's surface receptors (including cytokine receptors and homing receptors).

Note that a FRIL family member's ability to preserve progenitor cells by binding to a normally glycosylated FLT3 receptor on the progenitor cell is distinguishable from the progenitor cell's response when the normally glycosylated FLT3 receptor is bound by its natural ligand, the FLT3 ligand. For example, binding of the FLT3-ligand to a normally glycosylated FLT3 receptor on a progenitor cell preferably induces proliferation and/or differentiation of that FLT3 ligand-bound progenitor cell. In contrast, binding of a FRIL family member to a normally glycosylated FLT3 receptor on a progenitor cell induces that progenitor cell to enter a state of quiescence, thereby preserving that FRIL family member-bound progenitor cell.

As used herein, a “cytotoxic agent” is any chemical that kills cells. Included as cytotoxic agents are chemotherapeutics (e.g., cyclophosphatmide and cisplatin). Also included as cytotoxic agents of the invention are those chemicals, such as carbon tetrachloride (CCl₄) and dextran sulfate sodium (DSS) that kill some cells, but not others (e.g., CCl₄ produces heptocellular necrosis).

The invention is particularly useful where the cytotoxic agent is an agent that kills both neoplastic and normal cells. Because a FRIL family member will only bind to a normally glycosylated FLT3 receptor, only those cells expressing a normal glycosylated FLT3 cell will be protected from the activity of a cytotoxic agent. Thus, cancer cells, which are neoplastic and thus express aberrantly glycosylated FLT3 receptor, will not be bound by a FRIL family member, and thus will not be protected from the cytotoxic effects of a cytotoxic agent.

In various embodiments of the invention, the cytotoxic agent is a chemotherapeutic or a radiotherapeutic. In some embodiments, the progenitor cell is contacted with the FRIL family member molecule before the cell is contacted with the cytotoxic agent. In some embodiments, the progenitor cell is contacted with the FRIL family member molecule either after the cell is contacted with the cytotoxic agent or at the same time that the cell is contacted with the cytotoxic agent.

Preferably, the FRIL family member of the invention is purified. FRIL family member molecules are readily purified using standard techniques. Methods for purifying proteins are known in the art and include, without limitation, HPLC, SDS-PAGE, immunoprecipitation, recombinant protein production, affinity chromatography using specific antibodies, ion-exchange, size-exclusion, and hydrophobic interaction chromatography, or a combination of any of these methods. These and other suitable methods are described, e.g., in Marston, “The purification of eukaryotic proteins expressed in E. coli,” in DNA Cloning, Glover D. M., ed., Volume III, IRL Press Ltd., Oxford, 1987; Marston and Hartley, “Solubilization of protein aggregates,” pp. 266-267 in Guide to Protein Purification, Deutscher M. P., ed., Academic Press, San Diego, 1990; Laemmli, U. K., Nature 227:680-685, 1970; Ausubel et al., Current Protocols in Molecular Biology, John Wiley & Sons, New York, N.Y., 1999; U.S. Pat. No. 6,084,060; and Gowda et al., J. Biol. Chem. 269:18789-18793, 1994. A FRIL family member can also be purified by binding to a mannose, which may be coupled on a sold support (e.g., a sepharose bead). Non-limiting sources from which naturally occurring FRIL family member molecules can be purified include Dolichos lab lab, Phaseolus vulgaris, Sphenostylis stenocarpa, Vigna sinensis, and Voandzeia subterranea.

Purification of a FRIL family member molecule from a legume is rapid and inexpensive, and results in a large amount of purified FRIL family molecule. FRIL family members are relatively abundant in legumes. For example, Dl-FRIL accounts for approximately 0.02% of the mass of hyacinth beans. By “purified” means a molecule, such as a protein (e.g., a FRIL family member molecule) or composition of that molecule, that is more free from other organic molecules (e.g., carbohydrates, nucleic acids, proteins, and lipids) that naturally occur with an impure molecule, and is substantially free as well of materials used during the purification process. For example, a protein is considered to be purified if it is at least approximately 60%, preferably at least approximately 75%, more preferably approximately at least 85%, most preferably approximately at least 90%, and optimally approximately at least 95% pure, i.e., free from other organic molecules with which it naturally occurs and free from materials used during the purification process. A FRIL family member molecule can be easily purified from legumes, such as hyacinth beans (which can be grown pesticide-free), by mannose-affinity chromatography or ovalbumin affinity chromatography, and is more than 100 times cheaper to produce than recombinant cytokines.

In certain embodiments of the first aspect of the invention, the progenitor cell is in an animal, including, without limitation, a domesticated animal, a laboratory animal or a human. By “domesticated animal” is meant an animal domesticated by humans, including, without limitation, a cat, dog, elephant, llama, horse, sheep, cow, pig, and goat. Also included as domesticated animals are non-mammals (e.g., turkeys and chickens). Non-limiting examples of a “laboratory animal” are non-human primates (e.g., chimpanzee, baboon), fish, frogs, worms, mice, rats, and rabbits. In certain embodiments where the progenitor cell is in an animal, the progenitor cell is contacted by administering the FRIL family member molecule to the animal. In certain embodiments, the FRIL family member molecule is administered to the animal with a pharmaceutically acceptable carrier. By “pharmaceutically acceptable carrier” is meant any inert carrier that is non-toxic to the animal to which it is administered and that retains the therapeutic properties of the compound with which it is administered (i.e., the FRIL family member).

Pharmaceutically acceptable carriers and their formulations are well-known and generally described in, for example, Remington's Pharmaceutical Sciences (18th Edition, Gennaro A., ed., Mack Publishing Co., Easton, Pa., 1990). Non-limiting exemplary pharmaceutically acceptable carriers are Hank's Balanced Salt Solution (HBSS) or physiological saline solution. Pharmaceutical formulations of the invention may employ any pharmaceutically acceptable carrier, depending upon the route of administration of the composition.

Compositions of FRIL family members are safe and efficacious for use as therapeutics. The gastrointestinal tracts of animals come in constant contact with lectins, such as FRIL family members, in raw and/or cooked vegetables and fruits. Many lectins pass through the gastrointestinal tract biologically intact (Pusztai, A., Eur. J. Clin. Nutr. 47: 691-699, 1993). Some lectins interact with the gut and are transported into the peripheral blood circulation. For example, a recent study found peanut agglutinin (PNA) in the blood of humans at levels of 1-5 μg/ml an hour after ingesting 200 g of raw peanuts (Wang et al., Lancet 352: 1831-1832, 1998). Antibodies to dietary lectins are commonly found in people at levels of approximately 1 μg/ml (Tchernychev and Wilchek, FEBS Lett. 397: 139-142, 1996). These circulating antibodies do not block carbohydrate binding of the lectins.

As demonstrated in the Examples below, because mice tolerate very high levels of compositions of FRIL family members, this may permit more effective protection of stem cells and progenitor cells by preventing their recruitment during aggressive dose intensification regimens aimed at increasing frequency and dosage levels of chemotherapy. As described below, while the biological activity of Dl-FRIL (i.e., FRIL from Dolichos lab lab) is similar to cytokines (ng/ml range), mice tolerated up to a 100 to 1,000-fold more Dl-FRIL than cytokines.

In a second aspect, the invention provides a method for protecting a progenitor cell in a patient against a progenitor cell-depleting activity of a therapeutic treatment in a patient, comprising administering a therapeutically effective amount of a FRIL family member molecule to the patient with the therapeutic treatment, wherein the progenitor cell in the patient is protected against the progenitor cell-depleting activity of the therapeutic treatment. In certain embodiments of the first aspect of the invention, the progenitor cell is in an animal, including, without limitation, a domesticated animal, a laboratory animal or a human. In some embodiments, the patient has cancer.

In accordance with the invention, by “therapeutically effective amount” is meant a dosage of a composition of a FRIL family member or pharmaceutical formulation comprising a composition of a FRIL family member that is effective either to alleviate and/or reduce either a condition whereby the patient's progenitor cells are depleted or to alleviate and/or reduce a progenitor cell-depleting activity of a therapeutic treatment (e.g., a chemotherapeutic). Preferably, such administration is systemic (e.g., by intravenous injection). When administered systemically, a therapeutically effective amount is an amount of between about 500 ng of the FRIL family member/kg total body weight and about 5 mg/kg total body weight per day. Preferably, a therapeutically effective amount is between about 500 ng/kg and 500 μg/kg total body weight of the FRIL family member per day. Still more preferably, a therapeutically effective amount is between about 5 μg/kg and 50 μg/kg total body weight of the FRIL family member per day. Most preferably, a therapeutically effective amount is an amount that delivers about 50 μg/kg total body weight of the FRIL family member per day.

According to the invention, administration of a FRIL family member to a patient being treated with the therapeutic treatment having a progenitor-cell depleting activity protects the patient's progenitor cells against the progenitor-cell depleting activity of the therapeutic treatment. A composition of a FRIL family member of the invention and pharmaceutical formulation comprising a composition of a FRIL family member of the invention may be administered patients having, or predisposed to developing, a condition whereby the patient's progenitor cells are depleted. Such a condition may be congenital. For example, the patient may have a plastic anemia.

The condition whereby the patient's progenitor cells are depleted may also be induced by a therapeutic treatment (e.g., treatment with chemotherapy). Thus, in certain embodiments of the second aspect of the invention, administration of a therapeutically effective amount of the pharmaceutical formulation to a patient prior to treatment of the patient with a therapeutic treatment having a progenitor cell-depleting activity alleviates the progenitor cell-depleting activity of the therapeutic in the patient. In certain embodiments of the second aspect, the therapeutic treatment is the administration of a radiotherapeutic, a chemotherapeutic, or a combination of a radiotherapeutic and a chemotherapeutic. For example, cancer patients are often treated with radiotherapeutics and/or chemotherapeutics that have progenitor cell-depleting activity. By “progenitor cell-depleting activity” is meant an activity of a therapeutic treatment whereby the progenitor cells in the patient being treated with the therapeutic treatment are depleted, either by killing the progenitor cells, by reducing the progenitor cells' ability to replicate, or by inducing the progenitor cells to undergo irreversible differentiation. Non-limiting examples of therapeutic treatments having progenitor cell-depleting activity are chemotherapeutic agents including, without limitation, cytarabine (Ara-C), doxorubicin (Dox), daunorubicin, docetaxel, topotecan, gemcitabine, etoposide, cisplatin, cyclophosphamide, paclitaxel, and 5-fluorouracil (5-FU). Additional non-limiting examples of therapeutic treatments having progenitor cell-depleting activity are radiotherapeutic agents including, without limitation, Rhenium-188 HEDP, Cobalt-60, and phosphorus-32 compounds.

The invention encompasses any and all mechanisms by which the FRIL family member molecule is able to protect progenitor cells from this progenitor cell-depleting activity of the therapeutic treatment. In one non-limiting mechanism, the FRIL family member molecule temporarily halts proliferation by a progenitor cell, thereby allowing the progenitor cell to escape the toxicity of the therapeutic treatment.

Moreover, in various embodiments of the second aspect of the invention, the patient is administered the FRIL family member molecule before administration of the therapeutic treatment. Thus, in one non-limiting example of the invention, the patient is administered a therapeutically effective amount of a FRIL family member (or a composition and/or pharmaceutical formulation comprising a FRIL family member) between about 5 days before the patient receives treatment with a therapeutic treatment (e.g., a chemotherapeutic) having a progenitor cell-depleting activity to about 2 hours prior to treatment with the therapeutic treatment, wherein the therapeutically effective amount of a composition and/or pharmaceutical formulation of the FRIL family member is administered daily. In accordance with the invention, preferably the patient is administered a therapeutically effective amount of a composition and/or pharmaceutical formulation comprising a therapeutically effective amount of a FRIL family member between about 2 days before the patient receives treatment with a therapeutic treatment (e.g., a chemotherapeutic) having a progenitor cell-depleting activity to about 1 day prior to treatment with the therapeutic treatment (where the therapeutically effective amount of a composition and/or pharmaceutical formulation of a FRIL family member is administered daily).

It will be understood that once the patient starts to receive treatment of a therapeutic treatment having a progenitor cell-depleting activity, the therapeutically effective amount of a composition and/or pharmaceutical formulation comprising a FRIL family member may be different from the therapeutically effective amount of the a composition and/or pharmaceutical formulation comprising a FRIL family member that the patient received prior to receiving the therapeutic treatment.

In some embodiments, the patient is administered the FRIL family member molecule after administration of the therapeutic treatment, or is administered the FRIL family member molecule at the same time the patient is administered the therapeutic treatment.

In certain embodiments, administration of a FRIL family member of invention to a patient together with administration to the patient of a therapeutic treatment having a progenitor cell-depleting activity enables treatment of the patient with a higher dosage of the therapeutic treatment. The higher dosage of the therapeutic treatment may be accomplished by either an increased dose of the therapeutic treatment and/or an increased duration of treatment with the therapeutic treatment. For example, a child diagnosed with childhood Acute Myelogenous Leukemia (AML) is typically initially treated for the first seven days with daunorubicin at 45 mg/m² on Days 1-3 plus Ara-C at 100 mg/m² for 7 days plus GTG at 100 mg/m² for 7 days. The same child pretreated with a composition in accordance with this aspect of the invention may be able to tolerate a higher dosage (i.e., higher dose and/or prolonged treatment period) of any or all of these chemotherapeutics. Such an increase in dosage tolerance of a therapeutic treatment (e.g., a chemotherapeutic) having a progenitor cell-depleting activity in a cancer patient is desirable since a higher dosage may result in the destruction of more cancerous cells. Dosages for the therapeutic treatment (e.g., daunorubicin) are known to any ordinarily skilled physician, and will vary based on the weight and age of the patient (see, e.g., Physician's Desk Reference, Medical Economics Co. 2000).

In accordance with the invention, FRIL family member may be administered by any appropriate means. For example, a FRIL family member may be administered to an mammal within a pharmaceutically-acceptable diluent, carrier, or excipient, in unit dosage form according to conventional pharmaceutical practice. Administration may begin before the mammal is symptomatic for a condition whereby the patient's progenitor cells are depleted. For example, administration of a FRIL family member molecule to a cancer patient may begin before the patient receives radiotherapy and/or chemotherapy treatment, or after the patient receives radiotherapy and/or chemotherapy treatment, but before the patient shows signs of illness due to the radiotherapy and/or chemotherapy treatment (e.g., hair loss, weight loss, digestion problems).

Any appropriate route of administration of a FRIL family member molecule of the invention may be employed, including, without limitation, parenteral intravenous, intra-arterial, subcutaneous, sublingual, transdermal, topical, intrapulmonary, intramuscular, intraperitoneal, by inhalation, intranasal, aerosol, intrarectal, intravaginal, or by oral administration. Pharmaceutical formulations and/or compositions comprising a FRIL family member molecule may be in the form of liquid solutions or suspensions; for oral administration, formulations may be in the form of tablets or capsules; and for intranasal formulations, in the form of powders, nasal drops, or aerosols. The pharmaceutical formulations and/or compositions comprising a FRIL family member molecule may be administered locally to the area affected by a condition whereby the patient's progenitor cells are depleted. For example, where the patient's hematopoietic progenitor cells are affected (e.g., the patient develops anemia), a FRIL family member molecule may be administered directly into the patient's bone marrow. A FRIL family member molecule may also be administered systemically.

In a third aspect, the invention features a method for isolating a cell for repairing a tissue comprising contacting a population of cells with a FRIL family member molecule and isolating a cell specifically bound by the FRIL family member molecule, wherein the cell bound to the FRIL family member molecule is useful for repairing a tissue. In certain embodiments, the cell specifically bound by the FRIL family member molecule is induced to differentiate into a specific cell type by incubating the cell in vitro with a growth factor prior to injecting the cell into the recipient animal. Preferably, the cell and the recipient animal are from the same species. Preferably, the cell is isolated from the recipient animal or from an individual whose MHC matches the recipient animal (e.g., the recipient's identical twin). Preferably, the cells is from a human.

In one non-limiting example, bone marrow cells are collected from a patient suffering from cirrhosis of the liver. FRIL-binding cells are isolated from the bone marrow cells or from whole blood as described in the Examples below. (Note that another non-limiting method to isolate FRIL-binding cells is to stain the cells with a FRIL family member, followed by a detectably labeled anti-FRIL antibody (e.g., labeled with FITC or phycoerythrin), and then sorting those cells that bind to the detectable label. Anti-FRIL antibodies (monoclonal or polyclonal) can be generated by standard methods (see, e.g., Coligan et al., Current Protocols in Immunology, John Wiley & Sons Inc., New York 1994).) Some of the isolated FRIL-binding cells may be directly injected into the patient's liver, some of the cells may be cultured in vitro in the presence of growth factors that stimulate liver development, including, but not limited to hepatocyte growth factor (HGF), and members of the TGFβ superfamily. In addition, some of the FRIL-binding cells are cryopreserved and stored in liquid nitrogen according to standard methods. The cells can be stored directly after isolation for later use as undifferentiated cells, or they can be cultured in the presence of growth factors that stimulate liver development prior to storage.

In certain embodiments of the third aspect of the invention, the population of cells preferably includes a progenitor cell. In certain embodiments, the population of cells is whole blood, umbilical cord blood, fetal liver cells, or bone marrow cells.

In certain embodiments of this aspect of the invention, the cell bound by the FRIL family member molecule is a progenitor cell. In certain embodiments, the progenitor cell is a messenchymal progenitor cell, a hematopoietic stem cell, a hair follicle progenitor cell, a skin progenitor cell, a liver progenitor cell, or a gastrointestinal progenitor cell.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLE 1

### Enrichment of Peripheral Blood Mononuclear Cells

The following standard procedure was used to prepare peripheral blood mononuclear cells (PBMCs) from either umbilical cord blood or peripheral blood and to remove red blood cells. (Human umbilical cord blood (CB) samples were obtained from full term deliveries.) This general protocol has been described (see, e.g., Proc. Natl. Acad. Sci. 92:10119-10122,1995; Clin. Lab. Haem. 20:341-343, 1998; Vox Sang. 76:237-240, 1999)

Blood was collected under sterile condition (100 μl are saved for cell count) and the total volume of blood was recorded. Next, hetastarch (stock is 6% in 0.9% NaCl, commercially available from Abbott, NDC 0074-7248-03, 500 ml) was added to blood to a 1.2% final concentration, and mixed well. The blood was next allowed to sit at room temperature (approximately 25° C.) for 45 minutes. The color and clarity of the top layer was noted during this time.

During this 45 minute “sitting time,” an initial cell count was made (using a hemacytometer). To do this, 180 μl of 2% acetic acid was mixed with 20 μl of the saved cells (leaving 80 μl) for a 1:10 dilution, and the cells were counted.

After the 45 minute “sitting time”, the leukocyte-enriched top layer was transferred to a new 50 ml conical tube, and the leukocytes washed twice with degassed HAEM. (HAEM is HBSS (Life Technologies, 14025-076)+0.1% AIM V Media (Life Technologies 12055-083)+1 mM EDTA(Sigma E-7889 Lot #110K89271), and is degassed by shaking for five minutes.) To wash, the volume of the cells was brought up to 40 ml with degassed HAEM, then the cells were centrifuged for 10 minutes at 400×g at 11° C. in a Beckman GS-6R centrifuge, and the supernatant aspirated.

After washing, the cell pellet was resuspended in 1 mL HAEM, and the remaining red blood cells lysed. To lyse the red blood cells, 9 mL of NH₄Cl solution (0.72% NH₄Cl, 10 mM EDTA, StemCell Technologies, Inc., Vancouver, Canada) was added to 1 ml of cell suspension, the tube mixed and placed on ice for five minutes.

Next, the cells are washed twice in degassed HAEM (volume is raised to 40 ml with degassed HAEM, the cells centrifuged for 5 min at 1300 RPM and 11° C., then the supernatant is aspirated). The cell pellet was then resuspended in 20 ml degassed HAEM. Finally, the remaining cells were counted, and were ready to be used for either FRIL functional studies or sorting using FRIL binding.

### EXAMPLE 2

### FRIL-Binding Cell Selection to Isolate Progenitor Cells

The following standard procedure was used to isolate progenitor cells from the peripheral blood mononuclear cells prepared as described in Example 1.

Using sterile techniques, the cells from Example 1 were centrifuged for 5 minutes at 1300 RPM at 11° C. (cells are in 20 ml from Example 1). The supernatant was aspirated, and the cells resuspended in 500 μl with 450 μl degassed HAEM (generally, it was assumed that there were 50 μl of cells and excess HAEM in the cell pellet).

Next, 50 μl of biotinylated FRIL (bFRIL; stock at 1 μg/ml) was added the 500 μl of cells and mixed (creates 1:10 dilution bFRIL to volume of cells). (To make biotinylated FRIL, FRIL was purified according to standard methods (see U.S. Pat. No. 6,084,060; Mo et al., Glycobiology 9: 173-179, 1999; Kollet et al., Exp. Hematol. 28: 726-726, 2000; Hamelryck et al., J. Molec. Biol. 299: 875-883, 2000) and biotinylated according to the Pierce Chemical Co. protocol). The bFRIL/cell mixture was incubated on ice for 30 minutes.

Next, the bFRIL/cell mixture volume was raised to 40 ml with degassed HAEM, and the cells were centrifuged cells for 5 min@1300 RPM and 11° C. After the supernatant was aspirated, the cells were resuspended in 500 μl of degassed HAEM. Next, Strepavidin (SA) beads (Miltenyi 100-18-101) were added to the cells at a concentration of 5 μl beads per per 10⁷ cells. The bead-cell mixture was mixed and place on ice for 5 minutes. Next, the volume of the cells was raised to 2 ml with 1.5 ml degassed HAEM.

In the meantime, a MidiMACS separation apparatus (Miltenyi 423) and a MACS LS⁺ separation column (Miltenyi 424-01) was prepared. The column was set in the magnet with a pre-filter (Pre-separation filters 30 um; Miltenyi 414-07) with a 50 ml conical test tube placed below the column. Three mls of degassed HAEM were run through the pre-filter to start collecting the “FRIL negative cells”.

Immediately after preparing the column, while there was still a meniscus of HAEM in the column, the cell sample was transferred to the pre-filter. Occasionally, the pre-filter needed to be jostled slightly to release a vacuum created.

After the sample was run through the column, 2 ml degassed HAEM was pipetted into the test tube where the cells were to wash the test tube and collect the excess cells. The 2 ml was then pipetted into the pre-filter.

Next, 2 more mls of degassed HAEM was pipetted through the pre-filter before the column ran dry from the last wash. After the pre-filter had no more drops, the pre-filter was jostled, and then removed. The column was next washed with a series of washes: 3 washes were run with 2 ml of degassed HAEM, and then 2 washes with 5 ml of degassed HAEM. The column was never allowed to become dry, and the tip of the column was always dripping.

Next, the column was removed from the magnet and placed on top of a 15 ml conical test tube labeled “FRIL pos cells” which was positioned directly beneath the tip of the column, so that the tip was inside the test tube. This 15 ml conical tube was used to catch the cells if they splattered when removing the column from the magnet.

Immediately after removing the column, 2 ml of degassed HAEM were pipetted into the column, and immediately the HAEM was plunged forcefully through the column into the test tube with the sterile plunger provided with the column. The final volume was between 2 and 2.5 ml.

Next, the plunged cells were centrifuged for 5 min at 1300 RPM and 11° C., and the cell pellet resuspended in 2 ml fresh HAEM, and counted (using Trypan Blue to count the number of living cells versus dead cells). For a viability count, the total number of living cells was divided by the total number of alive and dead cells, and then multiplied by 100 for a percentage. These FRIL-binding cells were now ready to use.

Typically, from cord blood, approximately 0.3% of the cells bound to FRIL.

### EXAMPLE 3

### Analysis of FRIL-Binding Progenitor Cells

FRIL-binding progenitor cells isolated as described in Example 2 were analyzed by cell staining (followed by FACS analysis on a Becton Dickson FACScan) according to standard methods (see, e.g., Coligan et al., Current Protocols in Immunology, John Wiley & Sons Inc., New York 1994) using commercially available antibody (e.g., FITC labeled anti-human CD34 commercially available from Becton Dickinson, phycoerythrin (PE)-labeled anti-human CD38 commercially available from Coulter, Miami Fla. USA, and PE-labeled anti-human CD135 (anti-Flt3) commercially available from Beckman Coulter).

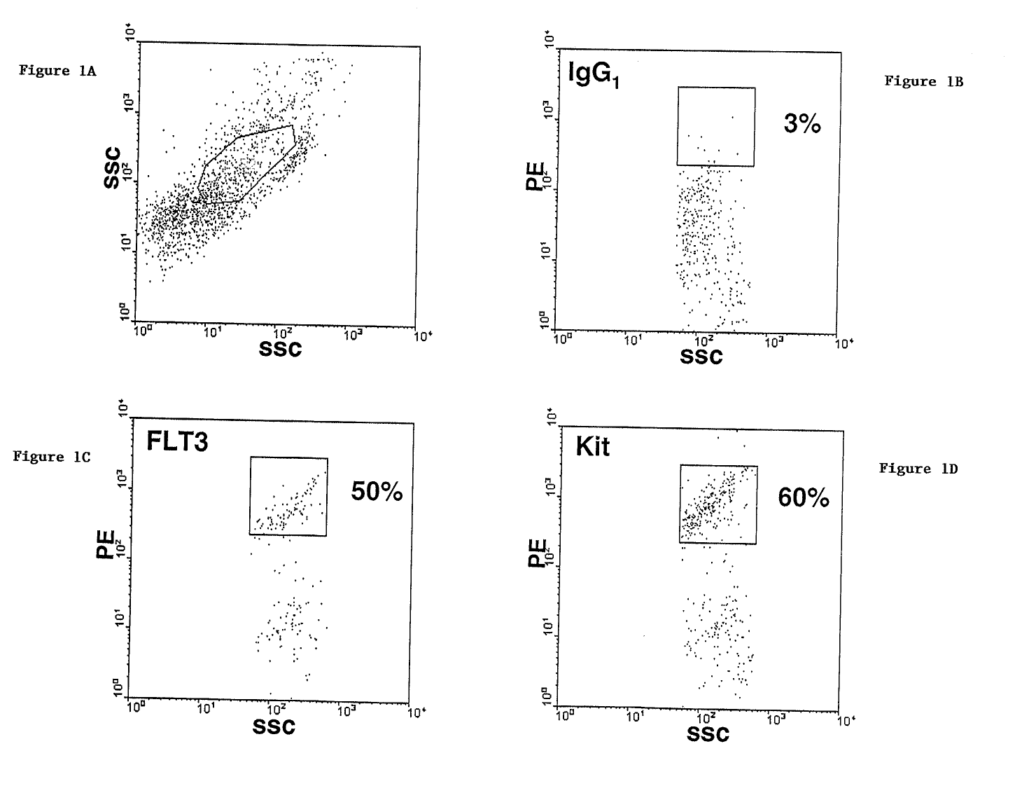

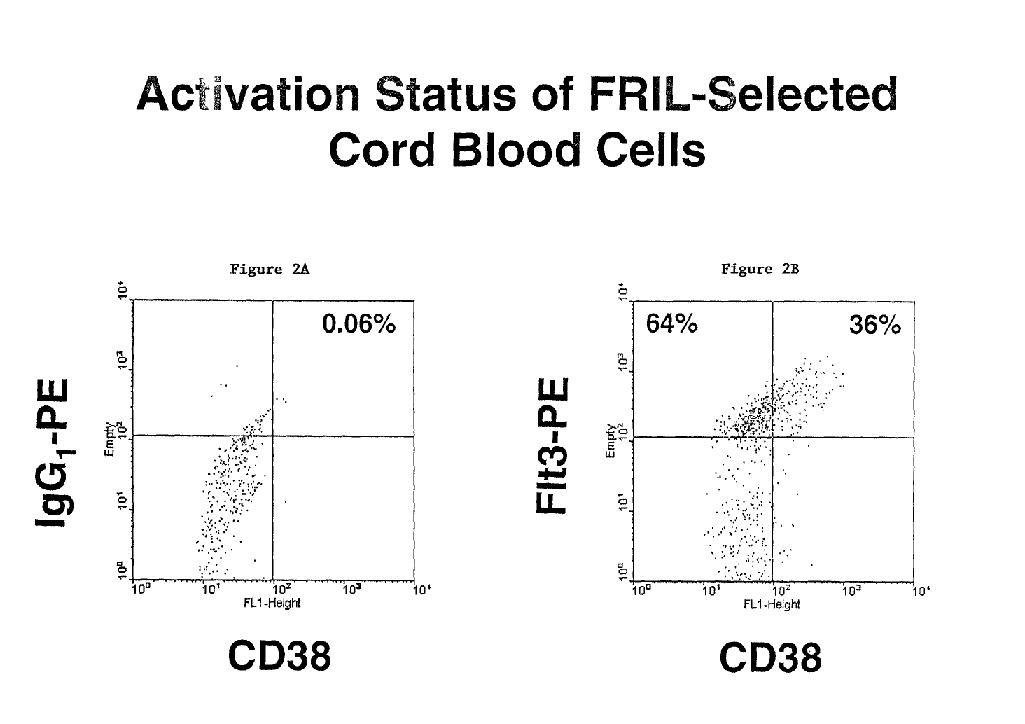

As shown in FIGS. 1A-1D, the FRIL-binding cells isolated as described in Example 2 were found to be approximately 50% positive for FLT3 receptor expression (FIG. 1C), and 60% positive for Kit receptor expression (FIG. 1D). In addition, as shown in FIG. 2B, of those cells that were FLT3 positive, 36% of the cells were positive for CD38, an activation marker found on blood leukocytes and lymphocytes. Thus, the majority of the FLT3 positive cells (64%) were not active.

(The fact that only 50% of the FRIL-binding cells are FLT3 positive indicated either that the anti-FLT3 antibody had a lower affinity for FLT3 receptor than FRIL_, that the FLT3 receptor was blocked by FRIL and so unable to be bound by the anti-FLT3 antibody, and/or that FRIL, in addition to binding the FLT3 receptor, binds to another receptor present on progenitor cells.)

The FRIL-binding cells, cells that did not bind FRIL (i.e., the cells that flowed through the column of Example 2 without sticking), and unsorted cord blood cells purified according to Example 1 were next plated in 24-well plates with approximately 10⁵ cells/well and incubated in 0.9% methylcellulose medium (StemCell Technologies) in the presence of endothelial stimulus (vascular endothelial growth factor (Biosource International, Camarillo, Calif.) at 100 ng/mL) or hematopoietic stimulus (agar conditioned medium, StemCell Technologies), for a colony-forming analysis. After two weeks in culture, the number of colonies was scored by microscopy.

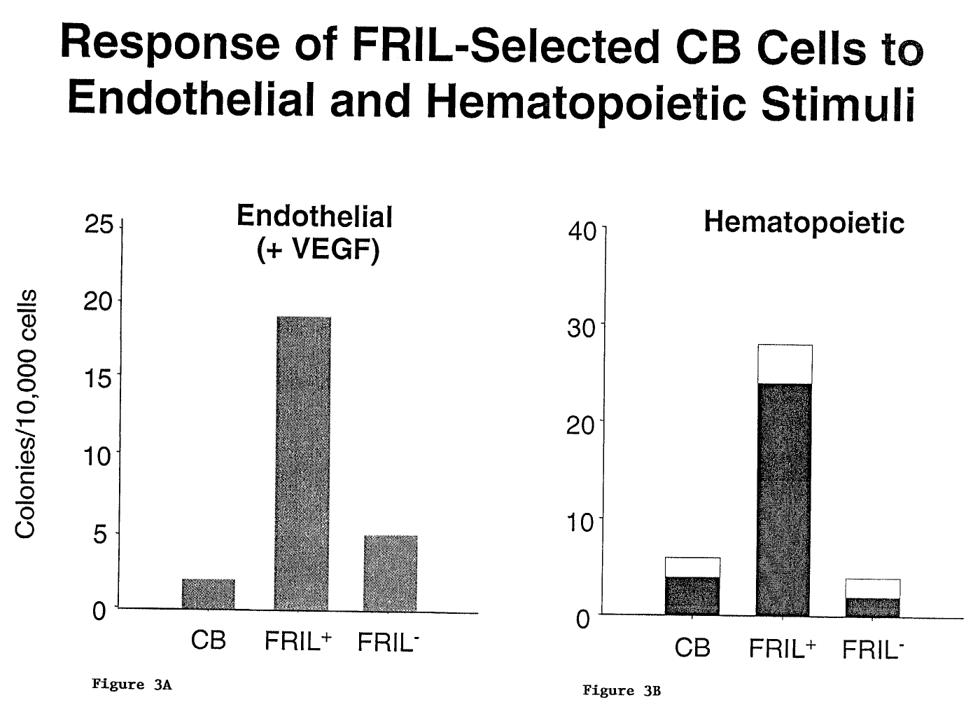

As shown in FIGS. 3A and 3B, unsorted cord blood cells and cells that did not bind to FRIL (“FRIL−” formed a relatively few number of colonies when incubated in the presence of either enthothelial or hematopoietic stimuli. In contrast, the FRIL-binding cells (“FRIL+”) formed a large number of colonies in response to either enthothelial or hematopoietic stimuli. The solid bars in the endothelial cultures represent colonies containing a cluster of cells of uniform morphology. The solid bars in the hematopoietic cells consist of colonies containing either myeloid or erythroid cells; the open bars indicate mixed colonies, derived from a more primitive progenitor, containing both myeloid and erythroid colonies.

Thus, these results demonstrated that FRIL-binding cells were very early progenitor cells which retained an ability to differentiate into both endothelial cells and hematopoietic cells depending upon the type of stimulus they were incubated in.

### EXAMPLE 4

### FRIL-Binding Cells are Similar to CD34⁺CD38^{-/low} Cells

A comparison was made of FRIL-binding cells to peripheral blood mononuclear cells (PBMCs) and CD34⁺ cells.

The PBMCs were prepared as described in Example 1.

CD34⁺ cells were prepared as follows: the cells prepared as described in Example 1 were enriched for CD34⁺ cells using the mini MACS separation kit (Miltenyi Biotec, Bergisch Gladbach, Germany) according to the manufacturer's instructions. The purity of the enriched CD34⁺ cells was 60-80% using one column.

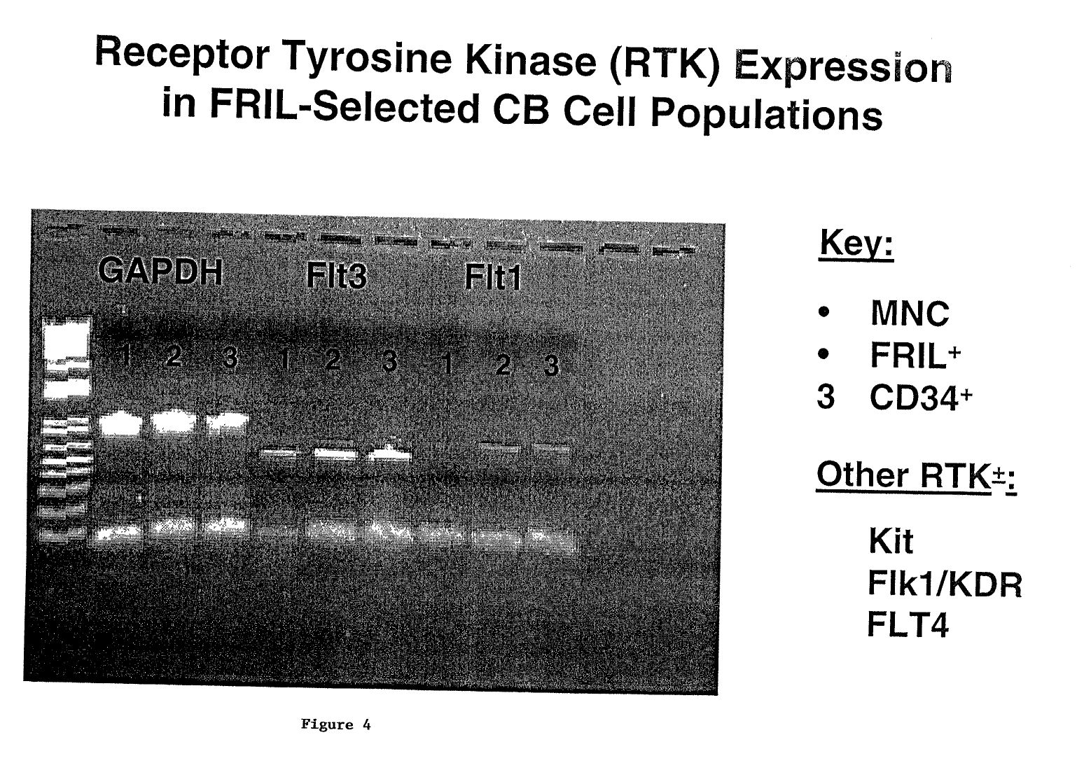

Total RNA was prepared from equivalent numbers of each cell type and assayed by RT-PCR for the presence of transcripts encoding the Flt-1 receptor, the Flt-3 receptor, or a control gene (GADPH) used to ascertain equivalent cell numbers. PCR products were resolved by agarose gel electrophoresis and visualized with ethidium bromide according to standard methods (see, e.g., Ausubel et al., supra). As can be seen in FIG. 4, all cell types tested (lane 1, PMNC; lane 2, FRIL-binding cells; lane 3, CD34+ cells), expressed equal levels of GAPDH. In contrast, PMNC's expressed lower levels of both the FLT3 and FLT1 receptors.

### EXAMPLE 5

### Mice Tolerate High Levels of Dl-FRIL

Previous studies have shown that the biological activity of FRIL in vitro is similar to chemokines and cytokines (ng/ml range) (see, e.g., Moore et al., Biochim. Biophys. Acta 25027: 1-9, 2000; Kollet et al., Exp. Hematol. 28: 726-736, 2000; Colucci et al., Proc. Natl. Acad. Sci. USA 96: 646-650, 1999; Mo et al., Glycobiology 9: 173-179, 1999). Chemokines and cytokines are often toxic at high levels. Accordingly, studies were performed to determine whether or not FRIL is similarly toxic at high levels.

To do these studies, Dl-FRIL (the FRIL family member from Dolichos lab lab) was purified according to standard methods (see U.S. Pat. No. 6,084,060; Mo et al., supra; Kollet et al., supra; Hamelryck et al., J. Molec. Biol. 299: 875-883, 2000). Dl-FRIL was administered intravenously to mice over a 3-log dose range of 0.006-1 mg/kg (0.32-20 μg/mouse). Dl-FRIL was well tolerated and these mice have subsequently received 2 monthly challenges of Dl-FRIL without any observable short- or long-term adverse effects.

Protocols to test chemoprotective properties of cytokines in mice typically involve daily pre-treatment (bolus or continuous delivery) regimens of 4-10 days before starting chemotherapy. Using this framework as a starting point, Dl-FRIL was injected at 5 mg/kg (100 μg/mouse) intravenously daily for 4 days. No gross adverse effects have been observed in over 150 mice treated with this dose regimen.

In another study, mice were given 4-8 doses of FRIL (0.05-5 mg/kg/dose) administered daily by intravenous or subcutaneous routes over the course of a week without any observable adverse reactions.

In another study designed to get a preliminary indication of the upper limit of FRIL, 4 BALB/c mice (2 male and 2 females, aged 5 months) received a single bolus intraperitoneal injection (to accommodate 1 ml volume) of FRIL at 500 mg/kg and were monitored for survival for 48 hours. This dose of FRIL, which was 100 fold higher than the typical high dose of 5 mg/kg, was lethal to 3 of the 4 mice (LD₇₅). The surviving mouse's weight decreased by approximately 15% in the first 2 day and returned to normal by day 4. The surviving mouse's blood counts were in the normal range 3 days after injection of FRIL. The results demonstrate that even a very large dose of Dl-FRIL was not completely toxic.

### EXAMPLE 6

### FRIL Family Member Proteins Protect CB MNC from the Toxicity of Chemotherapy Drugs

To determine whether or not a FRIL family member protein could protect progenitor cells from the toxicity of chemotherapy drugs, cord blood mononuclear cells (CB mnc) were collected as previously described (see Kollet et al., supra). CB mnc were then cultured in ninety-six well tissue culture plates (Corning Inc., Corning, N.Y.) at a concentration of 200,000 cells/mL in a volume of 0.1 mL of serum-defined medium (AIMV, commercially available from Life Technologies). Thus, there were 20,000 cells total per well.

Dl-FRIL (the FRIL family member from Dolichos lab lab) was purified according to standard methods (see U.S. Pat. No. 6,084,060; Mo et al., supra; Kollet et al., supra; Hamelryck et al., supra). Dl-FRIL was added at a concentration of 10 ng/ml or 100 ng/ml (with no addition as a control), together with cytarabine (Ara-C), doxorubicin (Dox), cisplatin, or 5-fluorouracil (5-FU) over a 5-log dose range. Cultures were incubated in humidified chambers without medium changes for up to 29 days. Viable cells were determined after 5 days of culture by XTT(2,3-bis[Methoxy-4-nitro-5sulfophenyl]-2H-tetrazolium-5 carboxanilide inner salt) (Diagnostic Chemicals Ltd, Charlottetown, Prince Edward Island, Canada), which is a tetraformazan salt cleaved by actively respiring cells (Roehm et al., J. Immunol. Methods 142: 257-265, 1991). Proliferation and cell survival was quantitated spectrophotometrically using a Vmax kinetic plate reader (Molecular Devices Corp., Mountain View, Calif.), and recorded as either relative activity (units/mL) or as specific activity (units/mg).

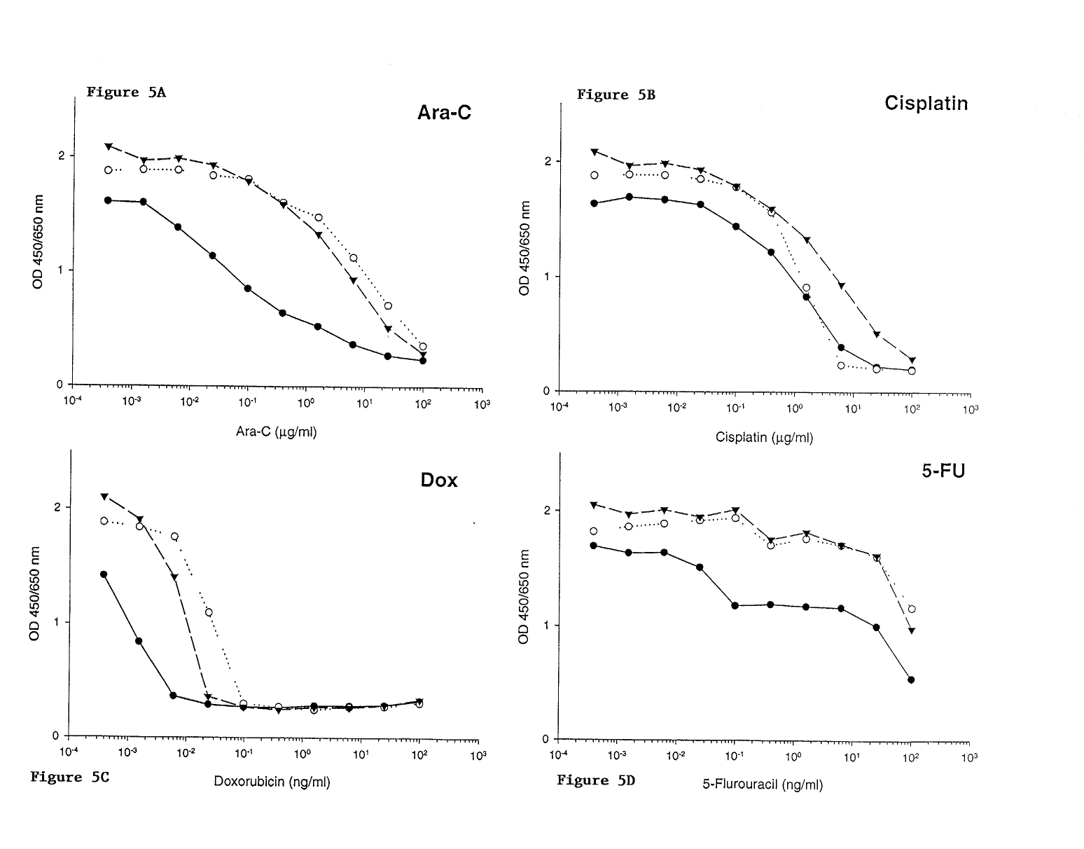

As shown in FIGS. 5A-5C, cultures containing Dl-FRIL (either at 10 ng/ml (solid triangles) or 100 ng/ml (open circles)) showed a decrease susceptibility to cytarabine (Ara-C) (FIG. 5A), cisplatin (FIG. 5B). or doxorubicin (Dox) (FIG. 5C) by 10- to 10,000-fold. As shown in FIG. 5D, the presence of FRIL in the 5-FU cultures increased cell viability over a large dose range. The differences observed between the dose shift of Ara-C, Dox, and cisplatin by Dl-FRIL as compared to 5-FU may be explained by recent reports demonstrating that 5-FU acts via an RNA mechanism rather than as a DNA-specific drug (Bunz et al., J. Clin. Invest. 104:263-269, 1999).

### EXAMPLE 7

### Dl-FRIL Protects Mice from 5-FU Induced Death in the Critical First Two Weeks

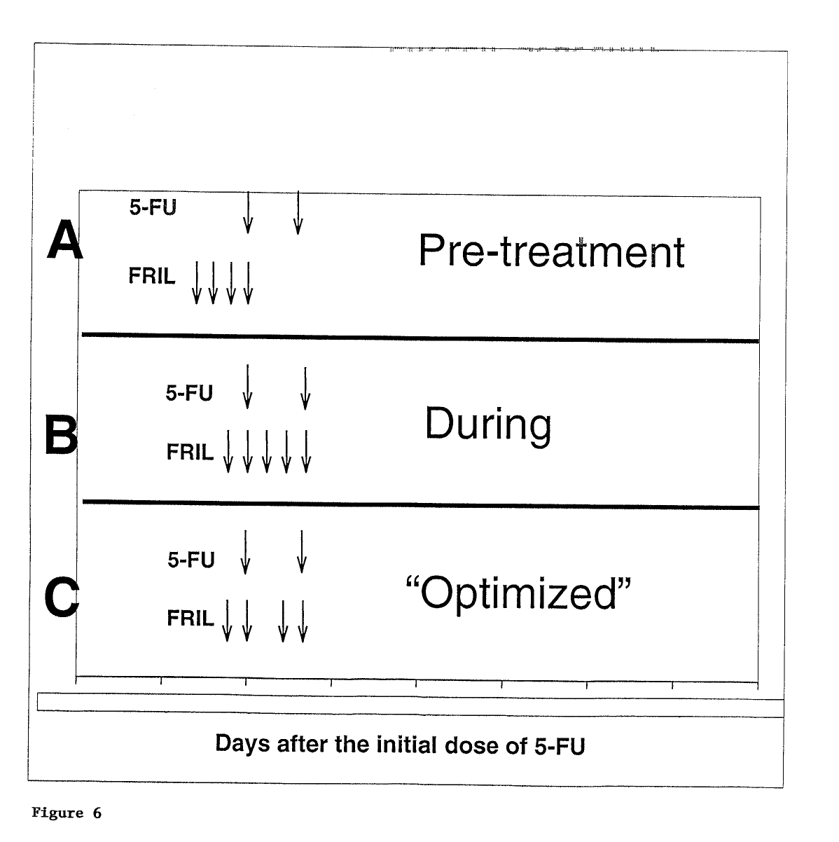

A dose regimen was established to determine whether FRIL protects mice from death resulting from hematopoietic toxicity of 5-fluorouracil (5-FU). This murine 5-FU chemoprotection model was based on studies by Lerner and Harrison (Exp. Hematol. 18:114-118, 1990) and de Haan et al. (Blood 87:4581-4588., 1996). As shown in FIGS. 6A-6C, three different dose regimens for administering Dl-FRIL with 5-FU were performed. Eventually, to do the studies in mice, a modification combining the pretreatment dose regimen (FIG. 6A) and the “optimized” dose regimen (FIG. 6C) was used. This dose regimen for Dl-FRIL described above (5 mg/kg×4 days or 5 days) was selected based on the requirement of treating animals with cytokines for one or several days prior to starting chemotherapy and because Dl-FRIL-treated mice easily tolerated doses (5 mg/kg) that were 10- to 100-fold greater than that used for cytokines (see Example 1). Since FRIL and cytokines act on progenitors in the same concentration range (ng/ml), this pretreatment dose regimen was used to test whether Dl-FRIL can protect mice from 5-FU administered at 2 intervals in the first week:

Dl-FRIL was purified from Dolichos lab lab as described above in Example 1.

In a first study, two groups of weight-matched BALB/c mice (10 mice/group) were injected intravenously (i.v.) with either 0.2 mL of Dl-FRIL in HBSS (500 μg/mL, i.e., 100 μg per injection) (Group 1) or with 0.2 mL of Hank's Balanced Salt Solution (HBSS; Group 2) daily for five consecutive days. Two hours after the second injection of Dl-FRIL or HBSS (day 0), mice were injected intraperitoneally (i.p.) with 5-FU (150 mg/kg). In addition, one day after the final injection of Dl-FRIL or HBSS (day 5), mice received a second i.p. injection of 150 mg/kg 5-FU. No mice died from a single dose of 5-FU.

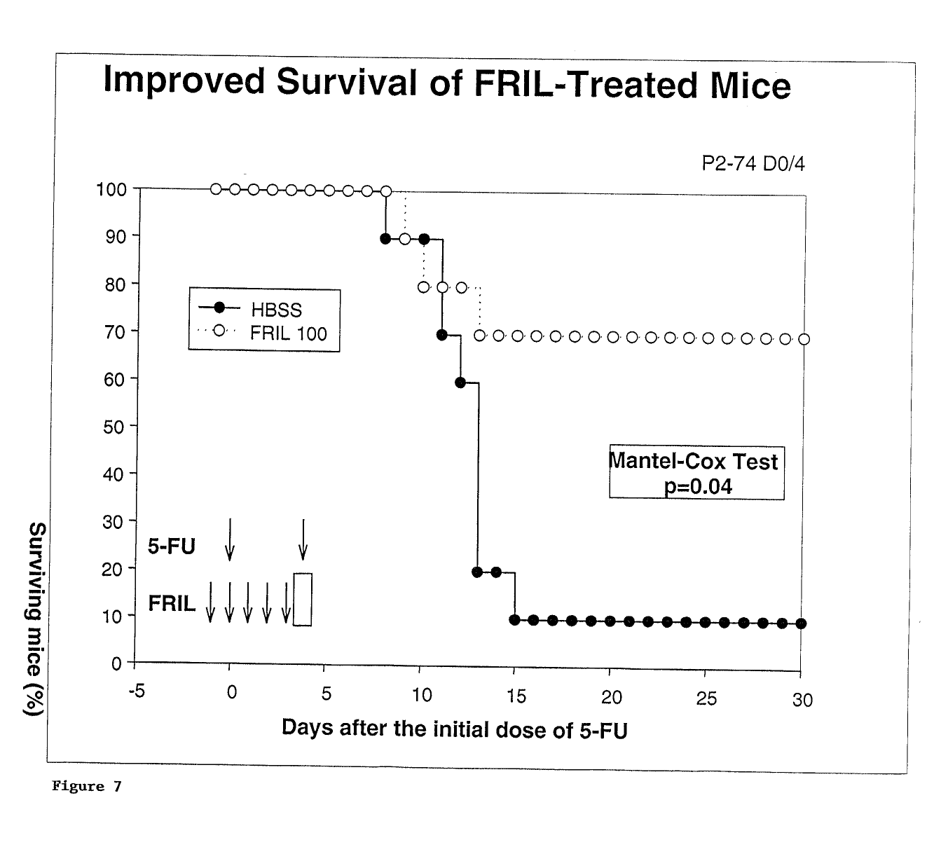

As shown in FIG. 7, seven out of ten mice in the Dl-FRIL-treated group of mice (Group 1; open circles), survived two weeks after treatment with two doses of 5-FU. In contrast, only one out of ten mice in the HBSS-treated group of mice (Group 2; closed circles in FIG. 7) survived the two doses of 5-FU two weeks after receiving the first 5-FU dosage.

In a second study, weight-matched BALB/c mice (10 mice/group) were injected intravenously with 0.2 mL of HBSS containing 100 μg DI-FRIL (Group 1); 0.2 mL of HBSS containing 10 μg Dl-FRIL (Group 2); 0.2 mL of HBSS containing 1.0 μg Dl-FRIL (Group 3); 0.2 mL of HBSS containing 0.1 μg Dl-FRIL (Group 4); or with 0.2 mL of HBSS (Group 5) daily on days −1, 0, 4, and 5. Two hours after injection with Dl-FRIL or HBSS on days 0 and 5, mice were injected intraperitoneally (i.p.) with 5-FU (150 mg/kg). No mice died from a single dose of 5-FU.

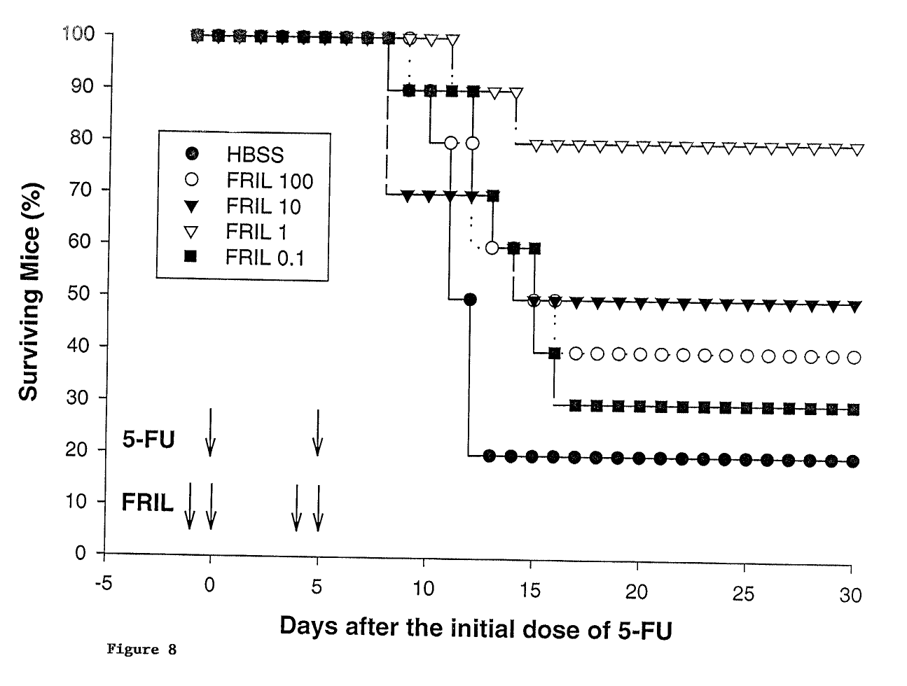

As shown in FIG. 8, two to three weeks after the first 5-FU treatment, mice in Group 3 (open triangles), which had received 1.0 μg Dl-FRIL on days −1, 0, 4, and 5 showed the highest survival (eight out of ten survived), whereas only two of the ten mice that received no Dl-FRIL (Group 5; closed circles in FIG. 8) survived past two weeks after the first 5-FU treatment. Treatment of mice with 0.1 μg of FRIL (Group 4; closed squares in FIG. 8) resulted in only 30% survival. As FIG. 8 shows, in this example and FRIL dosage timing, higher doses of FRIL at 10 μg and 100 μg protected 50% and 40% of mice, respectively, possibly because the progenitors were held in a dormant state too long and consequently delayed restoration of the bone marrow

In another study involving two separate Experiments (16 week old males or eight week old females), weight-matched BALB/c mice (10 mice/group) were injected intravenously with either with 0.2 mL of Dl-FRIL (500 mg/mL, i.e., 100 μg per injection) or 0.2 mL of HBSS daily for 4 days. Two hours after the final treatment of Dl-FRIL, mice were injected intraperitoneally with 5-FU (150 mg/kg). Groups of mice received a second dose of 5-FU (150 mg/kg) at either day 3 (D0/3) or day 5 (D0/5). As before, no mice died from a single dose of 5-FU.

As shown below in Table I, in Experiment 1 (16 week old males), when the mice were pretreated with FRIL and received a second dose of 5-FU at day 3, three out of ten mice survived, as compared to the death of all ten mice that received only HBSS pre-treatment and 5-FU at days 0 and 3.

TABLE I

| | | 5-FU Dose Interval | | 5-FU Dose Interval | |
|---|---|---|---|---|---|
| | | D0/3 | | D0/5 | |
| Exp. | Mice | FRIL | HBSS | FRIL | HBSS |
| 1 | Males, 16 wk | 3/10 | 0/10 | N.T. | N.T. |
| 2 | Females, 8 wk | 0/10 | 0/10 | 4/10 | 1/10 |

Improved survival of mice pretreated with FRIL (5 mg/kg × 4 days) prior to undergoing 5-FU dose intervals of d0/3 and d0/5.

Similarly, in Experiment 2 of Table I, although no mice survived when treated with 5-FU at days 0 and 3, when the mice were treated with 5-FU at days 0 and 5, the group of mice that received FRIL pre-treatment had a 40% survival rate as compared to the 10% survival rate observed in the HBSS pretreated group.

In sum, these studies demonstrated that mice being administered 5-FU showed much higher survival rates when they are also administered a FRIL family member protein, whether the administration is prior to administration of the first 5-FU dosage or at the same time as the administration of both the first and the second 5-FU dosages.

### EXAMPLE 8

### FRIL Prevents Wasting of Mice in the Two-Dose 5-fluorouracil Chemoprotection Model.

The mice in Example 3 that were administered both FRIL and 5-FU appeared healthy and normal while those mice administered HBSS and 5-FU appeared sick. Accordingly, studies were performed to determine if FRIL could prevent wasting that is typically seen in chemotherapy-treated animals.

To do this, BALB/c mice (females, aged 7-8 weeks, 10 mice/group) were injected subcutaneously with either 0.2 mL of FRIL (100 μg/mL, i.e., 20 μg total) or 0.2 mL HBSS the day before and 1 hour before treatment with 5-FU (150 mg/kg, i.p.) on days 0 and 5. That is, mice were administered FRIL or HBSS on days −1, 0, 4, and 5. Mice were weighed daily.

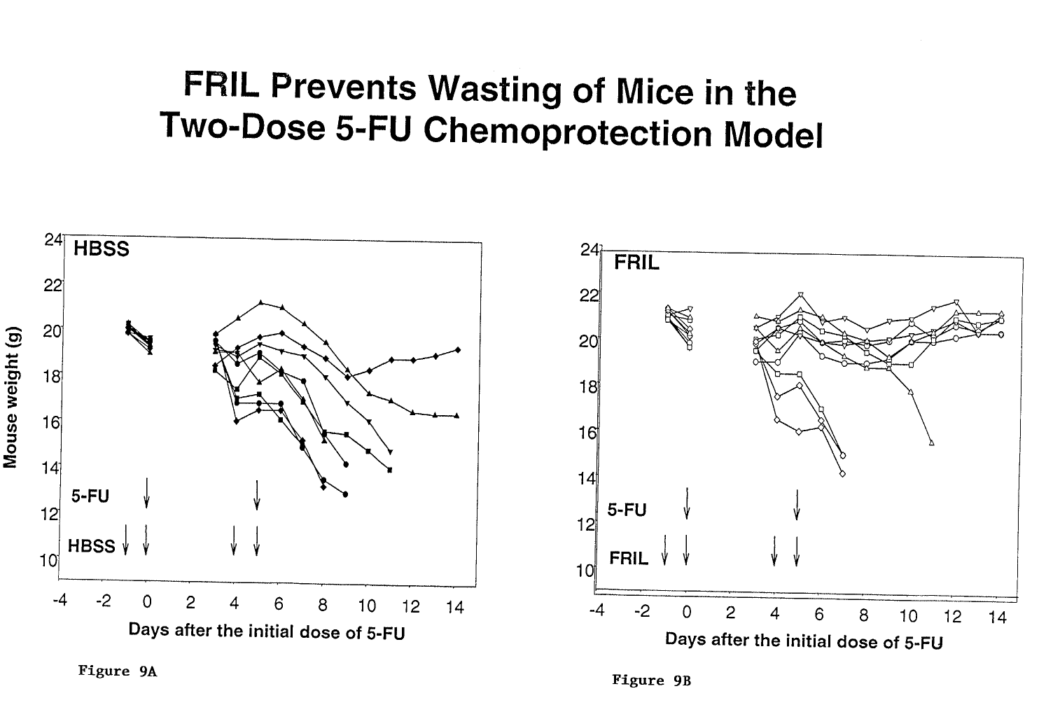

As shown on FIGS. 9A and 9B (a representative experiment), the life-sparing chemoprotective properties of FRIL extend beyond the bone marrow to protect other tissues and organs damaged by chemotherapy. The surviving FRIL-treated mice did not show the hallmark chemotherapeutic toxicity signs of weight loss and lethargic behavior, in contrast to the surviving HBSS only treated mice. These results demonstrate that FRIL protects bone marrow-derived stem cells and the progenitors needed to restore the health of not only blood cell production but also of other tissues and organs such as the liver, heart, nervous system, and gastrointestinal tract damaged by chemotherapy.

### EXAMPLE 9

### Establishment of the Maximal Tolerated Dose (MTD) of FRIL

Athymic nude mice (commercially available from the Jackson Laboratory, Bar Harbor, Me) are used to establish MTD for a single intravenous injection of FRIL.

To do this, FRIL is administered to 4 groups (doses of 240, 120, 60, and 30 mg/kg) of 2 athymic female mice each. The nice are observed daily for survival and morbundity, and are weighed on days 1, 5, 9, and 14. The study is terminated on day 14. The study is repeated at higher or lower dose levels, depending on whether the mice tolerate all dose levels or whether all dose levels kill the mice. In this way, the MTD in athymic nude mice is determined.

In another study, FRIL is administered intravenously (i.v.) on days 1-6 or days 1, 2, 5, 6, 9, and 10. The nice are observed daily for survival and morbundity, and are weighed on days 1, 5, 9, 13, 17, 21, 25, and 30. The study is terminated on day 30. The study is repeated at higher or lower dose levels, depending on whether the mice tolerate all dose levels or whether all dose levels kill the mice. In this way, the MTD in athymic nude mice is determined.

### EXAMPLE 10

### Treatment Schedule of FRIL with a Cytotoxic Agent

Next, a dose-range finding study for FRIL using treatment schedules appropriate for combining with four clinically approved agents is performed. The study will consist of eight groups of four athymic female mice each(four dose levels/treatment schedule). FRIL is administered intravenously (i.v.) 1 day before and 1 hour before each treatment with a cytotoxic agent. The cytotoxic agent is administered using either daily injections for 5 days (for paclitaxel) or 3 injections separated by 4-day intervals (for 5-fluorouracil, doxorubicin, and cyclophosphamide). The dose levels are determined by the results of the preliminary dose-range finding study).

### EXAMPLE 11

### A FRIL Family Member has Chemoprotective Properties With Widely Used Cell Cycle-Active Chemotherapeutics

After establishing the optimal dose regimen of a FRIL family member, the FRIL family member's ability to protect mice from death by cytarabine (Ara-C) and doxorubicin is analyzed.

Initial dose regimens of cytarabine (Ara-C) and doxorubicin are as follows:

Doxorubicin—4 mg/kg as single bolus i.p. injection (Grzegorzewski et al., J. Exp. Med. 180: 1047-1057, 1994); Ara-C -300 mg/kg at time as an i.p. injection at 0 and 12 hours (Paukovits et al., Blood 77:1313-1319, 1991). Further studies are based on targeted clinical indication.

### EXAMPLE 12

### FRIL Maintains CD34⁺ Cells in a Quiescent State

Because FRIL-binding cells as prepared in Example 2 were very similar (although not identical) to CD34+ cells, the latter cells were used to determine what effects incubation with FRIL had on the cells. Note that because of the high affinity of FRIL to the cells, FRIL-binding cells as prepared in Example 2 were not used in this study, because the cells had already bound FRIL, and so were already rendered quiescent.

CD34⁺ cells were prepared by sorting the cells prepared as described in Examples 1 for CD34 expression using the mini MACS separation kit (Miltenyi Biotec, Bergisch Gladbach, Germany) according to the manufacturer's instructions (see Example 5). The purity of the enriched CD34⁺ cells was 60-80% using one column.

Immediately after they were sorted (i.e., on day 0), the cells permeabilized (incubated with 0.1% triton-X), stained with propidium iodide (PI), and subjected to FACscan analysis using FACSort (Becton Dickinson, San Jose, Calif.). The cell cycle stage of a cell was determined by the amount of nucleic acid the cell had (i.e., how PI was uptaken by the cell).

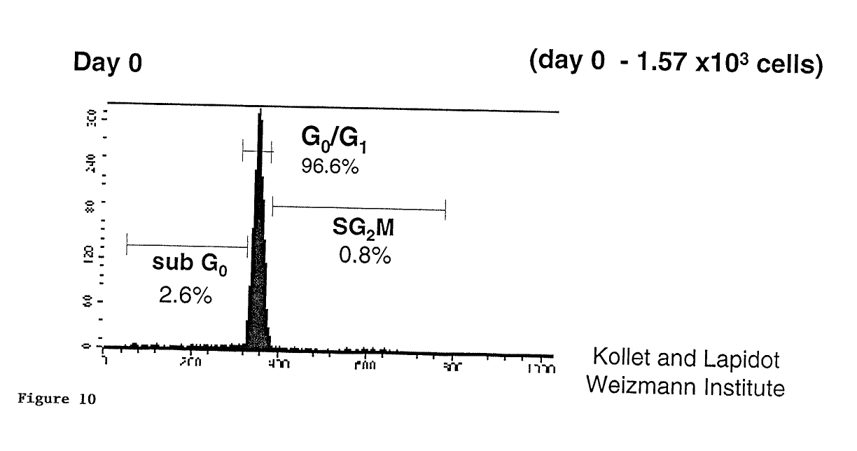

As shown on FIG. 10, the vast majority of the cells (96.6%) were quiescent (i.e., in the G₀/G₁ stage of the cell cycle) on the day they were sorted.

Next, the sorted cells were cultured in 24 well plates (2-4×10⁵ cells/well) in the presence of Dl-FRIL (40 ng/mL), SFR6 (stem cell factor and Flt-3 ligand, 100 ng/mL (R&D Systems Inc., Minneapolis, Minn.); rhIL-6, 50 ng/Ml; sIL6R, 1280 ng/mL (Inter-Pharm Laboratories, Ness Ziona, Israel); and FRIL, 40 ng/mL (ImClone Systems Inc., New York, N.Y.)), or SFG36 (stem cell factor, 300 ng/mL; Flt-3 ligand, 300 ng/mL; G-CSF, 50 ng/mL (R&D Systems Inc., Minneapolis, Minn.); IL-3 and IL-6, 10 ng/mL (R&D Systems Inc., Minneapolis, Minn.)), and the number of cycling cells was assessed after 3, 6, and 10 days of incubation.

TABLE II
Number of Cycling Cells (× 10⁻³)

| Days in Culture | FRIL | SFR6 | SFG36 |
|---|---|---|---|
| 3 | 6.3 | 6.9 | 71.0 |
| 6 | 5.9 | 30.7 | 132.6 |
| 10 | 3.2 | 45.0 | 491.6 |

As shown in Table II, while incubation in FRIL maintained the CD34⁺ cells in a non-cycling quiescent state even after ten days of incubation, incubation in SFR6 or SFG36 stimulated the CD34⁺ cells to enter the cell cycle.

### EXAMPLE 13

### FRIL Trumps Cytokine Stimulation

The ability of FRIL to maintain mononuclear cells was next compared to the ability of cytokines to stimulate their entry into the cell cycle. Accordingly, mononuclear cells (as prepared according to Example I) were incubated in the presence of both FRIL (from 0 to 1000 ng/ml) and agar conditioned medium containing cytokines (StemCell Technologies) for five days.

TABLE III

| | Number of Mononuclear | Progenitor Frequency | |
|---|---|---|---|
| FRIL (ng/ml) | Cells (× 10⁻⁴) | Myeloid | Erythroid |
| 0 | 120 | 38 +/− 7 | 63 +/− 32 |
| 0.1 | 115 | 21 +/− 7 | 50 +/− 7 |
| 1 | 120 | 16 +/− 12 | 38 +/− 7 |
| 10 | 70 | 77 +/− 12 | 103 +/− 6 |
| 100 | 55 | 44 +/− 15 | 85 +/− 12 |
| 1000 | 20 | 90 +/− 85 | 150 +/− 42 |

As shown in Table III, FRIL acted in a dominant manner over cytokines when the cells were stimulated with both. Thus, incubation in 1000 ng/ml of FRIL. while preventing the mononuclear cells from proliferating, led to the greatest frequency of myeloid and erythoid progenitor colonies.

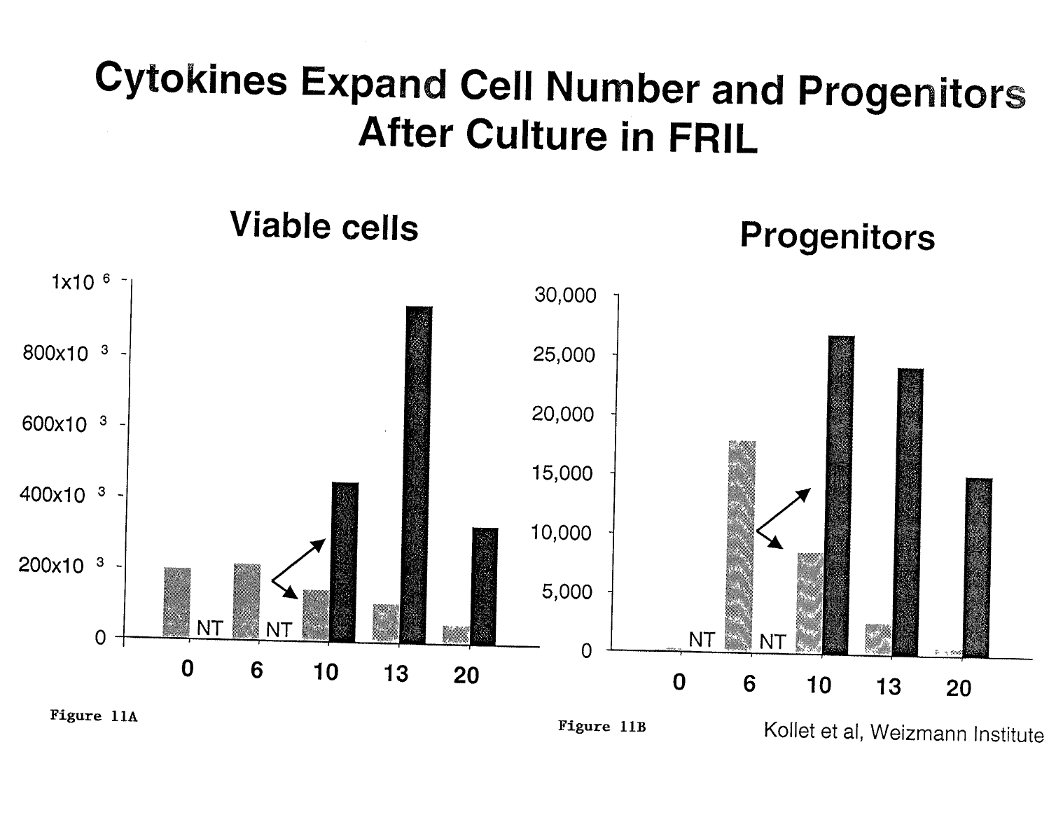

Furthermore, after incubation in FRIL, the cells were clearly viable and able to respond to cytokines. In a separate experiment, cells were cultured in FRIL (40 ng/mL) for 6 days. At day 6, the cells were harvested and washed free of exogenous FRIL, and were split equally in cultures containing either FRIL or cytokine cocktails. After four additional days of culture, cells were harvested and counted (day 10). Similar treatments of FRIL-treated cells were carried out after ten days of incubation with FRIL followed by three days of cytokine stimulation (day 13), and thirteen days of culture followed by seven days of cytokine stimulation (day 20). As shown in FIGS. 11A and 11B, the cells treated only with FRIL (gray bars) did not expand following incubation with FRIL, while both viable cells and progenitor cells increased in number when cells were treated with cytokines following incubation with FRIL (black bars).

### EXAMPLE 14

### Dl-FRIL Protects Mice From Dextran Sulfate Sodium (DSS)-Induced Colitis

Oral administration of dextran sulfate sodium (DSS) induces acute and chronic colitis in mice. The mechanism of DSS-induced colitis is under investigation. Current research is focused on the toxic effects on colonic epithelium, alterations in luminal bacterial flora, and activation of macrophage inflammatory responses (Mahler et al., Am. J. Physiol. 274: G544-G551, 1998).

To determine if a FRIL family member protein protects mice from DSS-induced colitis, FRIL is purified according to standard methods and is administered before and during DSS-administration in mice, and colitis is analyzed by histological analysis.

Weight-matched C3H/HeJBir or NOD/SCID/B2 mice (10 mice/group, equal number of males and females, aged 6-8 weeks, The Jackson Laboratory, Bar Harbor, Me.)) are injected subcutaneously (subQ) with either 0.2 mL of Dl-FRIL in HBSS (500 μg/mL, i.e., 100 μg per injection) (Group 1) or with 0.2 mL of Hank's Balanced Salt Solution (HBSS; Group 2) daily for seven consecutive days. Two hours after the second injection of Dl-FRIL or HBSS (day 0), experimental colitis is induced by giving the mice a 3.5% (wt/vol) DSS (molecular weight of 36,100-45,000); TB Consultancy, Uppsala, Sweden) in acidified drinking water ad libitum for five days. Once DSS administration is stopped, and mice receive acidified drinking water alone for 16 days until necropsy on day 21. This dosage of DSS is empirically established to induce moderate to severe colitis while minimizing the mortality.

Mice are weighed daily and examined for evidence of diarrhea during exposure to DSS. Mice treated with HBSS control typically are found to have loose stools or diarrhea. In addition, in the HBSS control group, gross evidence of blood is frequently observed in the stools. Mice treated with FRIL typically are found to have fewer loose stools or diarrhea than those in the HBSS control group.

All mice are euthanized by CO₂ asphyxiation on day 21. The large intestine is collected, and the cecum is separated from the colon. Intestinal specimens are gently inflated with Fekete's acid-alcohol-Formalin fixative by intraluminal injection. The entire colon, including the rectum, is prepared as an intestinal roll by placing it on an index card and twisting it in concentric centrifugal circles around a central toothpick in the plane of the card. Samples are immersed in fixative overnight and then transferred to 70% ethanol. Tissues are processed, embedded in paraffin, sectioned at 5 μm, and stained with hematoxylin and eosin.

Two sections of each cecum and one section of each colon/rectum are coded with an accession number and are reviewed by a pathologist. Each lesion was scored for lesions based on severity, ulceration, hyperplasia, and area involved.

As compared to the HBSS treated colitis-induced mice, those colitis-induced mice that receive FRIL are found to show less intestinal damage (i.e., fewer lesions, severity, ulceration, hyperplasia, and less overall area affected).

### EXAMPLE 15

### Dl-FRIL Protects Mice From Carbon Tetrachloride (CCl₄)-Induced Liver Damage in Mice

Exposure of mice to CCl₄ produces heptocellular necrosis when oxidized to the active CCl₃ radical form. CCl₃ reacts with the cell membrane lipids, leading to damage of the membrane's integrity and eventual cell death.

To determine if a FRIL family member protein could protect mice from CCl₄-induced liver damage, FRIL is administered before and during CCl₄-administration in mice and liver damage is evaluated by analyzing serum cholyglycine levels, histological analysis (Biesel, K W et al., Br. J. Exp. Path., 65, 125-131, 1984).

Weight-matched BALB/c mice (10 mice/group, equal number of males and females, aged 6-8 weeks, The Jackson Laboratory) are injected subcutaneously (subQ) with either 0.2 mL of Dl-FRIL in HBSS (500 μg/mL, i.e., 100 μg per injection) (Group 1) or with 0.2 mL of Hank's Balanced Salt Solution (HBSS; Group 2) daily for seven consecutive days. Two hours after the second injection of DI-FRIL or HBSS (day 0), experimental liver damage is induced by injecting 0.3 ml of CCl₄ in olive oil (10% v/v). Four days after CCl₄ injection, mice are bled from the retro-orbital sinus, euthanized by CO₂ asphyxiation, and livers are removed and fixed in phosphate buffered 10% formalin and processed to 5 um thick paraffin sections. Sections are stained with hematoxylin and eosin. The levels of cholyglycine are measured by a radioimmunoassay using a commercially available kit according to manufacturer's instructions (Abbott Laboratories, North Chicago, Ill.). Serum levels are compared in samples obtained 1 day before CCl₄ injection and at days 1 and 4 after injection.

Lastly, histological analysis is performed to determine quantitative differences in the area of liver necrosis.

As compared to the HBSS treated liver damaged-induced mice, those liver damage-induced mice that receive FRIL are found to show less intestinal damage (i.e., fewer lesions, severity, ulceration, hyperplasia, and less overall area affected).

### EXAMPLE 16

### FRIL-binding Cells Are Totipotent and Useful for Tissue Repair

To determine if FRIL-binding cells are totipotent, fetal liver and/or fetal blood from an inbred strain of male mice (e.g., Balb/c from the Jackson Laboratory, Bar Harbor Me.) are sorted, as described above, for those able to specifically bind to FRIL-coated beads. To remove the cells from the beads, the bead-coated cells are flooded with a large excess of “free” FRIL (i.e., FRIL not coupled to a bead), and the beads removed by adherence to the magnet, thereby resulting in bead-free cells.

The cells are divided.

Some of the cells are incubated in a variety of growth factors, including factors that stimulate the development of muscle cells, kidney cells, skin cells, nervous tissue, epithelial cells, bone cells. Non-limiting examples of growth factors include bone morphogenetic protein-2, transforming growth factor-b (TGFb), fibroblast growth factor-1 (FGF-1), fibroblast growth factor-2 (FGF-2), ciliary neurotrophic factor (CNTF), nerve growth factor (NGF), hepatocyte growth factor (HGF), insulin-like growth factor (IGF), epidermal growth factor (EGF) and others (see, e.g. van den Berg et al. (2001) Clin. Orthoped. 391S:244-50; Tomita et al. (2000) J. Atheroscler. Thromb. 7: 1-7; Sleeman et al. (2000) Pharm. Acta. Helv. 74:265; and Wells (2000) Am. J. Physiol. Gastrointest. Liver Physiol. 279:845; Zaret (2000) 92:83.

The results show that a FRIL-selected cell is able to develop into a variety of tissues depending upon which growth factors it is incubated in.

Some of the cells are injected directly into neonatal female mice of the same inbred strain as the male fetuses from which the FRIL-selected cells were purified. In other words, the recipients differ from the donor only in that they lack a Y chromosome. The cells are injected into various body areas of the female mice (e.g., into the liver, into the blood stream, into the thymus). The mice are allowed to mature and, at various stages of life (e.g., at puberty, after giving birth), the mice are sacrificed and tissue sections made and stained for the presence of the Y chromosome. The results show that the injected cells are able to differentiate into any type of tissue depending upon where the cells were injected and/or where the cell migrated.

These studies demonstrate that FRIL-selected cells (i.e., capable of binding to FRIL) are totipotent and can be used for tissue repair.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method for protecting a progenitor cell against a cytotoxic agent comprising contacting the progenitor cell with a FRIL family member molecule and the cytotoxic agent, wherein the contacted progenitor cell is protected against cytotoxicity by the cytotoxic agent.

2. The method of claim 1, wherein the FRIL family member molecule is purified.

3. The method of claim 1, wherein the progenitor cell is in a tissue.

4. The method of claim 1, wherein the progenitor cell is a hematopoietic progenitor cell.

5. The method of claim 1, wherein the progenitor cell is selected from the group consisting of a messenchymal progenitor cell, a hematopoietic stem cell, a hair follicle progenitor cell, a skin progenitor cell, a liver progenitor cell, and a gastrointestinal progenitor cell.

6. The method of claim 1, wherein the progenitor cell is in an animal.

7. The method of claim 6, wherein the progenitor cell is contacted by administering the FRIL family member molecule to the animal.

8. The method of claim 7, wherein the FRIL family member molecule is administered to the animal with a pharmaceutically acceptable carrier.

9. The method of claim 6, wherein the animal is a human.

10. The method of claim 1, wherein the cytotoxic agent is selected from the group consisting of a chemotherapeutic and a radiotherapeutic.

11. The method of claim 1, wherein the progenitor cell is contacted with the FRIL family member molecule before the cell is contacted with the cytotoxic agent.

12. A method for protecting a progenitor cell in a patient against a progenitor cell-depleting activity of a therapeutic treatment in a patient, comprising administering a therapeutically effective amount of a FRIL family member molecule to the patient with the therapeutic treatment, wherein the progenitor cell in the patient is protected against the progenitor cell-depleting activity of the therapeutic treatment.

13. The method of claim 12, wherein the patient is human.

14. The method of claim 12, wherein the patient has cancer.

15. The method of claim 12, wherein the therapeutic treatment is selected from the group consisting of a radiotherapeutic, a chemotherapeutic, and a combination of a radiotherapeutic and a chemotherapeutic.

16. The method of claim 15, wherein the chemotherapeutic is selected from the group consisting of cytarabine, doxorubicin, cisplatin, daunorubicin, paclitaxel, cyclophosphamide, and 5-fluorouracil.

17. The method of claim 12, wherein the FRIL family member molecule is purified.

18. The method of claim 12, wherein the FRIL family member molecule is administered to the patient with a pharmaceutically acceptable carrier.

19. The method of claim 12, wherein the patient is administered the FRIL family member molecule before administration to the patient of the therapeutic treatment.

20. The method of claim 12, wherein the patient is administered the FRIL family member molecule after administration to the patient of the therapeutic treatment.

21. A method for isolating a cell for repairing a tissue comprising contacting a population of cells with a FRIL family member molecule and isolating a cell specifically bound by the FRIL family member molecule, wherein the cell bound to the FRIL family member molecule is useful for repairing a tissue.

22. The method of claim 21, wherein the population of cells includes a progenitor cell.

23. The method of claim 21, wherein the population of cells is from a human.

24. The method of claim 21, wherein the cell bound by the FRIL family member molecule is a progenitor cell.

25. The method of claim 21, wherein the progenitor cell is selected from the group consisting of a messenchymal progenitor cell, a hematopoietic stem cell, a hair follicle progenitor cell, a skin progenitor cell, a liver progenitor cell, and a gastrointestinal progenitor cell.

26. The method of claim 21, wherein the population of cells is selected from the group consisting of whole blood, umbilical cord blood, fetal liver cells, and bone marrow cells.
